(19) United States
(12) Patent Application Publication
(10) Pub. No.: US 2010/0183602 A1
(43) Pub. Date: Jul. 22, 2010

(54) Induction of Tolerogenic Phenotype in Mature Dendritic Cells

(75) Inventors: Jose M. Carballido Herrera, Basel (CH); Jan E. De Vries, Basel (CH); Ulf Korthaeuer, Basel (CH); Maria Grazia Roncarolo, Basel (CH); Silvia Adriana Gregori, Basel (CH)

Correspondence Address: NOVARTIS INSTITUTES FOR BIOMEDICAL RESEARCH, INC., 220 MASSACHUSETTS AVENUE, CAMBRIDGE, MA, 02139, US

(73) Assignee: Novartis AG

(21) Appl. No.: 12/663,431

(22) PCT Filed: Jun. 3, 2008
(86) PCT No.: PCT/EP08/56851

§ 371 Date: Dec. 7, 2009

(30) Foreign Application Priority Data
Jun. 5, 2007 (EP) 07109672.1

(51) Int. Cl. A61K 39/395; C07K 16/00; C12N 5/02; A61K 35/26; A61P 37/08; A61P 29/00

(52) U.S. Cl. 424/133.1; 530/387.3; 435/375; 424/93.7

(57) ABSTRACT

The present invention relates to the use of a CD45 binding molecule to modulate the function of Dendritic cells. In particular the present invention relates to the use of a CD45 binding molecule to induce tolerogenic dendritic cells, useful in the treatment of diseases or disorders such as autoimmune diseases, transplant rejection.

### FIELD OF THE INVENTION

The present invention relates to methods for modulating dendritic cell function. In particular, the invention relates to methods for generating tolerogenic dendritic cells and uses derivable from such methods. The present invention finds utility in for example the treatment and/or prophylaxis of pathological immune responses in a human, such as those immune responses associated with autoimmune disease, transplant rejection, psoriasis, inflammatory bowel disease, allergies and the like. The invention further relates to medicaments and pharmaceutical compositions obtainable from the methods defined herein.

### BACKGROUND

Discovery of new drugs able to suppress T-cell mediated responses could be beneficial for the treatment of several immuno-mediated diseases including acute organ rejection, graft-versus-host disease, autoimmune diseases, and chronic inflammation.

Bone marrow and organ transplantation are currently the treatment for a number of malignant and non-malignant disorders of both hematopoietic and non-hematopoietic origin and the end phase failure of most essential organs (liver, heart and lungs), respectively. However, rejection responses mediated by the immune system of the donor against the recipient, termed graft versus host disease (GvHD), remain a major cause of morbidity in bone marrow transplantation. Similarly, allograft rejection mediated by the recipient is a major hurdle to long-term graft survival after organ transplantation. Immonusuppressive drugs can successfully treat both GvHD and organ transplant rejection. However, these approaches require life-long treatment and suppress the entire immune system non-specifically, exposing patients to increased risks of infection and cancer. Furthermore, these non-specific therapies have only a limited beneficial impact on long-term graft survival (1).

Similarly, treatments of immune responses to self-antigens, which lead to destruction of peripheral tissues in autoimmune diseases, are currently based on modulation of inflammation and non-specific immunosuppression. These approaches are frequently not effective long-term due to the side effects of immunosuppression including infections and cancer, and high risk of disease relapse once the drug is withdrawn.

In chronic inflammatory diseases and in allergies an altered immune response to pathogenic and non-pathogenic antigens occurs. This may be due to an imbalance between effector and regulatory immune responses. Conventional anti-inflammatory or immunosuppressive therapies are often insufficient to restore this balance. Moreover, the benefit of these therapies is not long-lasting after drugs are withdrawn.

An alternative strategy to non-specific immuno-suppression is based on the induction of specific immune tolerance with the ultimate goal to down-regulate the pathogenic immune responses while keeping intact the mechanisms of host defense. Central tolerance occurs during T-cell ontogeny in the thymus and is mediated by clonal deletion of self-reactive T-cells, whereas peripheral T-cell tolerance is operational throughout life and is designed to control responses towards self-antigens and non-harmful foreign antigens such as food antigens. The normal processes that are generally involved in peripheral tolerance are: clonal deletion, clonal inactivation (anergy), cytokine-dependent immune-deviation, and suppression. The primary mediators of immune responses in allograft rejection, autoimmunity, and inflammation are T- and B-cells. Both of them require signaling not only through T- and B-cell receptors but also through costimulatory pathways (e.g. CD28 or CD80-86 and CD40/CD40L). Interference with these two signals during T-cell activation can induce anergy in CD4+ T-cell in vitro and in vivo as demonstrated in several preclinical models of transplantation (2-6). Promising drugs including non-mitogenic anti-CD3 mAb, anti-CD4 mAb and Campath-1H (anti-CD52) are being tested in transplanted patients. An example is a non-mitogenic anti-CD3 mAb, which has been used in kidney transplant trials without side effects (7, 8). Moreover, a single course of treatment with anti-CD3 mAbs modifies the progression of the autoimmune process in type 1 diabetes (9, 10), and in psoriatic arthritis (11). Recently, it has been demonstrated that in addition to its depleting effect (12), Campath-1H induces the expansion of T-regulatory cells (Tr cells) which ultimately suppress lethal GvHD in hu-PBL-SCID mice (13).

Blockade of the T-cell costimulatory targets CD28 and CD154 has been shown to induce a state of antigen-specific tolerance in murine pre-clinical models (4). Anti-CD154 mAb prevents acute renal allograft rejection (14) and promotes long-term allograft acceptance (15, 16) in non-human primates. Despite positive pre-clinical results, clinical trials testing anti-CD154 mAb as an immuno-modulatory agent in autoimmune diseases and transplantation were stopped due to thrombo-embolic complications (17). Alternative anti-CD154 mAb have been developed and it has been demonstrated that a short course of sirolimus and single donor-specific transfusion associated with anti-CD 154 mAb prolonged allograft survival in primates and induce tolerance (18, 19).

In addition to the above, the use of immunomodulatory cytokines, such as IL-10 and TGF-β may also induce a state of T-cell anergy. IL-10 plays a central role in controlling inflammatory processes, suppressing T-cell responses, and maintaining immunological tolerance (reviewed in (20)). IL-10 inhibits IFN-γ and IL-2 production by T-cells (21), it has anti-inflammatory effects inhibiting the production of pro-inflammatory cytokines, such as TNF-α, IL-1, IL-6, and chemokines, such as IL-8 and MIP1α, produced by activated antigen-presenting cells (APC), neutrophils, eosinophils, and mast-cells. Furthermore, IL-10 down-regulates the expression of MHC class II, co-stimulatory and adhesion molecules (22-24) on APC, and modulates their stimulatory capacity (25). Importantly, IL-10 is crucial for the differentiation of adaptive type 1 T regulatory (Tr1) cells (26). Tr1 cells are characterized by a unique cytokine secretion profile. Upon TCR activation they secrete high levels of IL-10, significant amounts of IL-5 and TGF-β, low levels of IFN-γ and IL-2, but no IL-4 (26). Ag-specific murine Tr1 cells can be generated in vitro by repetitive TCR stimulation in the presence of high doses of IL-10 (26). Furthermore, addition of IL-10 (and TGF-β in the mouse (27)) to mixed lymphocyte reaction (MLR) cultures (28) results in T-cell anergy. Importantly, allo-reactive Tr1 cell clones from healthy individuals have been originally isolated from IL-10-anergized CD4⁺ T-cells by limiting dilution (26).

The first suggestion that human Tr1 cells are involved in maintaining peripheral tolerance in vivo came from studies in severe combined immunodeficient (SCID) patients successfully transplanted with HLA-mismatched allogeneic stem cells. In the absence of immunosuppressive therapy, these patients do not develop GvHD. Interestingly, high levels of IL-10 are detected in the plasma of these patients and a significant proportion of donor-derived T-cells, which are specific for the host HLA antigens and produce high levels of IL-10, can be isolated in vitro (29). In a preclinical model of bone marrow transplantation, transfer of donor CD4⁺ T-cells anergized ex-vivo by host APC in the presence of IL-10 and TGF-β results in a markedly decreased GvHD in MHC class II mismatched recipients (27, 30).

Dendritic cells (DC) are highly specialized APC that classically initiate Ag-specific immune responses upon infection (31). This process involves the terminal maturation of DC, typically induced by agents associated with microbial infection. It is now clear that DC can be not only immunogenic but also tolerogenic. In steady state DC express an immature phenotype and can induce tolerance via deletion of Ag-specific effector T-cells and/or differentiation of Tr cells (32-36). Repetitive stimulation of naïve cord blood CD4+ T-cells with allogeneic immature DC results in the differentiation of IL-10-producing Tr cells (37), which suppress T-cell responses via a cell-contact dependent mechanism. We recently reported that peripheral blood nave CD4+ T-cells stimulated with allogeneic immature DC become increasingly hypo-responsive to re-activation with mature DC and after three rounds of stimulation with immature DC, they are profoundly anergic and acquire regulatory function. These T-cells are phenotypically and functionally similar to Tr1 cells since they secrete high levels of IL-10 and TGF-β, suppress T-cell responses via an IL-10- and TGF-β-dependent mechanism, and their induction can be blocked by anti-IL10R mAb (38).

Not only immature DC but also specialized subsets of tolerogenic DC can drive the differentiation of Tr cells. Maturation and function of DC can be regulated at different levels (39). Both pharmacological and biological agents have been shown to be capable of inducing tolerogenic DC (40). Immuno-modulatory cytokines such as IL-10 alone (41, 42), or in combination with TGF-β (43), as well as pro-inflammatory cytokines such as IFN-α (44, 45), and TNF-α (46) can drive the differentiation of tolerogenic DC and induce anergic T-cells with suppressive activity.

CD45 plays a crucial role in T-cell activation. Seven different CD45 isoforms, which differ in the size of their extracellular domains, while sharing identical cytoplasmic PTPase domains, are generated by alternative splicing. Although multiple CD45 isoforms can be simultaneously expressed by an individual lymphocyte, the higher and lower molecular weight (MW) isoforms are differentially distributed in subsets of CD4⁺ T-cells that have distinct functions and cytokine production profiles (47, 48). The expression of CD45 isoforms is highly regulated and dynamic. T-cell activation is associated with a decrease in the higher MW isoforms and concomitant up-regulation of the lower MW isoforms. The regulated expression of CD45 isoforms in distinct T-cell subsets highlights their biological importance. The PTPase activity of CD45 regulates multiple pathways in immune cells, including signal transduction through TCRs, integrins, and cytokine receptors (49, 50). The function of CD45 on TCR signaling is mostly stimulatory, whereas CD45 can have an inhibitory effect in cytokine signaling (49).

Antibodies targeting the RB isoform of CD45 in mice can induce long-term engraftment and donor-specific tolerance in murine renal, islet and heart allografts (51) (52). Anti-CD45RB mAbs causes a rapid shift in CD45 isoform expression from the high to low MW that is not associated to CD4+ T-cell depletion, but to increased CTLA-4 expression on CD4+ T-cells (53). The up-regulation of CTLA-4 has been demonstrated to be a requisite for anti-CD45RB-mediated tolerance (54). Anti-CD45RB mAbs not only induce anergy in CD4+CD25-effector T-cells but also CD4+CD25+Tr cells, which are required to maintain tolerance (55). The role of new thymic emigrants in tolerance induction by anti-CD45RB mAb has been recently investigated, and results are controversial. In islet transplantation, although treatment with anti-CD45RB in thymectomized mice significantly decreased early rejection, it did not modify the long-term tolerogenic effect (55). Conversely, in cardiac transplantation, thymectomy completely prevented anti-CD45RB-mediated tolerance. Interestingly, anti-CD45RB mAb induces tolerance via de-novo generation of antigen-specific CD4+ T-cells from the thymus (56).

In WO02/072832 (the entire content of which is incorporated herein by reference and to which the reader is specifically referred). CD45RO/RB binding molecules were shown to inhibit primary alloimmune responses in a dose dependent fashion as determined by in vitro MLR. It was further demonstrated that the CD45RO/RB binding molecules act directly on the effector T cells and modulate their function.

In view of the above, there is a need in the art to establish further methods and medicaments which facilitate the suppression of potentially pathological immune responses. The present invention seeks to address this issue by modulating immune cell function in such a way that harnesses the immune system's natural regulatory mechanisms.

### SUMMARY OF THE INVENTION

In one aspect the invention provides a method of modulating dendritic cell (DC) function, the method comprising exposing dendritic cells to a CD45RO/RB binding molecule.

In a second aspect the invention provides a method of modulating dendritic cell (DC) function, the method comprising exposing dendritic cells to a binding molecule, wherein said binding molecule comprises in sequence the hypervariable regions CDR1, CDR2 and CDR3, said CDR1 having the amino acid sequence Asn-Tyr-Ile-Ile-His (NYIIH), said CDR2 having the amino acid sequence Tyr-Phe-Asn-Pro-Tyr-Asn-His-Gly-Thr-Lys-Tyr-Asn-Glu-Lys-Phe-Lys-Gly (YFNPYNHGTKYNEKFKG) and said CDR3 having the amino acid sequence Ser-Gly-Pro-Tyr-Ala-Trp-Phe-Asp-Thr (SGPYAWFDT); or wherein said molecule is a direct equivalent thereof.

In a preferred embodiment, the binding molecule comprises:

a) a first domain comprising in sequence the hypervariable regions CDR1, CDR2 and CDR3, said CDR1 having the amino acid sequence Asn-Tyr-Ile-Ile-His (NYIIH), said CDR2 having the amino acid sequence Tyr-Phe-Asn-Pro-Tyr-Asn-His-Gly-Thr-Lys-Tyr-Asn-Glu-Lys-Phe-Lys-Gly (YFNPYNHGTKYNEKFKG) and said CDR3 having the amino acid sequence Ser-Gly-Pro-Tyr-Ala-Trp-Phe-Asp-Thr (SGPYAWFDT); and
b) a second domain comprising in sequence the hypervariable regions CDR1′, CDR2′ and CDR3′, CDR1′ having the amino acid sequence Arg-Ala-Ser-Gln-Asn-Ile-Gly-Thr-Ser-Ile-Gln (RASQNIGTSIQ), CDR2′ having the amino acid sequence Ser-Ser-Ser-Glu-Ser-Ile-Ser (SSSESIS) and CDR3′ having the amino acid sequence Gln-Gln-Ser-Asn-Thr-Trp-Pro-Phe-Thr (QQSNTWPFT); or direct equivalents thereof.

Preferably the binding molecule is a chimeric, humanized or fully human monoclonal antibody.

Thus in one embodiment, the binding molecule is a humanised monoclonal antibody. In another embodiment, the binding molecule is a fully human monoclonal antibody.

Examples of suitable binding molecules for use in the present invention include, but are not limited to:
(a) A binding molecule comprising a polypeptide of SEQ ID NO: 1 and/or a polypeptide of SEQ ID NO:2;
(b) A binding molecule comprising a polypeptide of SEQ ID NO: 3 and/or a polypeptide of SEQ ID NO:4;
(c) A binding molecule which is a humanized antibody comprising a polypeptide of SEQ ID NO: 9 or of SEQ ID NO: 10 and/or a polypeptide of SEQ ID NO: 7 or of SEQ ID NO: 8; and
(d) A binding molecule which is a humanized antibody comprising a polypeptide of SEQ ID NO: 31 or of SEQ ID NO: 32 and/or a polypeptide of SEQ ID NO: 7 or of SEQ ID NO: 8

In one embodiment the method of modulating DC function is performed in vitro. In such cases, the DC may be obtained from a biological sample (i.e. ex vivo) or generated in vitro for example through obtaining a population of monocytes and inducing the monocytes to undergo in vitro differentiation into DC. In the case of the latter, the source of monocytes may be a biological sample.

In one embodiment, the method of modulating DC function comprises obtaining a source of immature DC and inducing maturation of the immature DC in the presence of a binding molecule as defined herein.

The methods of modulating DC function find use in inducing a tolerogenic phenotype in DC. In one embodiment, the method of modulating DC function further comprises the step of exposing the DC in vitro to a population of T-cells (e.g. allogeneic T-cells) so as to induce a tolerogenic phenotype in said T-cells. Such tolerogenic T-cells are also referred to herein as Tr cells.

The methods of modulating DC function also find use in the manufacture of medicaments/pharmaceutical compositions, e.g., for the treatment and/or prophylaxis of disease associated with autoimmune disease, transplant rejection, psoriasis, inflammatory bowel disease and allergies. In a preferred embodiment, the methods, uses and medicaments/pharmaceutical compositions of the invention find use in the treatment of psoriasis and/or transplant rejection in humans (such as allogenic transplantation e.g. pancreatic islet transplantation, in humans).

Accordingly, in a further aspect the invention provides a method of treatment and/or prophylaxis of disease associated with autoimmune disease, transplant rejection, psoriasis, inflammatory bowel disease and allergies, comprising administering to a human subject in need of such treatment and/or prophylaxis an effective amount of DC which has been modulated by exposure to a binding molecule as defined herein.

In another aspect the invention provides a method of treatment and/or prophylaxis of disease associated with autoimmune disease, transplant rejection, psoriasis, inflammatory bowel disease and allergies, comprising:

(a) obtaining from a human donor a population of monocytes;
(b) inducing in vitro differentiation of said monocytes so as to generate a source of DC;
(c) exposing the DC to a binding molecule as defined herein such that the DC become tolerogenic; and
(d) administering to a human recipient in need of such treatment and/or prophylaxis an effective amount of the tolerogenic DC.

In a further aspect of the invention, there is provided a method of treatment and/or prophylaxis of disease associated with autoimmune disease, transplant rejection, psoriasis, inflammatory bowel disease and allergies, comprising:

(a) obtaining from a human donor a population of DC;
(b) exposing the DC to a binding molecule as defined herein such that the DC become tolerogenic; and
(c) administering to a human recipient in need of such treatment and/or prophylaxis an effective amount of the tolerogenic DC.

In one embodiment, the donor and recipient of the above aspects are the same individual. In an alternative embodiment, the donor and recipient are different individuals, such that the DC are allogeneic with respect to the recipient.

A further aspect of the invention provides a method of treatment and/or prophylaxis of disease associated with autoimmune disease, transplant rejection, psoriasis, inflammatory bowel disease and allergies, comprising:

(a) obtaining from a first human donor a population of monocytes;
(b) inducing in vitro differentiation of said monocytes so to generate a source of DC;
(c) exposing the DC to a binding molecule as defined in any one of claims 1 to 10 as set forth below such that the DC become tolerogenic;
(d) exposing the tolerogenic DC to a population of T-cells obtained from a second human donor such that the T-cells become tolerogenic; and
(e) administering to a human recipient in need of such treatment and/or prophylaxis an effective amount of the tolerogenic DC and/or the tolerogenic T-cells.

In yet a further aspect, the invention provides a method of treatment and/or prophylaxis of disease associated with autoimmune disease, transplant rejection, psoriasis, inflammatory bowel disease and allergies, comprising:

(a) obtaining from a first human donor a population of dendritic cells;
(b) exposing the dendritic cells to a binding molecule as defined in any one of claims 1 to 10 as set forth below such that the dendritic cells become tolerogenic;
(c) exposing the tolerogenic dendritic cells to a population of T-cells obtained form a second human donor such that the T-cells become tolerogenic; and
(d) administering to a human recipient in need of such treatment and/or prophylaxis an effective amount of the tolerogenic dendritic cells and/or the tolerogenic T-cells.

In one embodiment, the first donor and/or the second donor are the same individual as the recipient. The first donor may be the same individual as the second donor or, alternatively, the first and second donors may be different such that the DC from the first donor and the T-cells from the second donor are allogeneic with respect to one another. In one embodiment, the first donor and recipient are the same individual and the second donor is a different individual. This embodiment finds particular use in the treatment of GvHD wherein the second donor provides the graft tissue for transplantation to the recipient/first donor.

Preferably in the above methods the DC are immature DC prior to their exposure to the CD45RO/RB binding molecule and the DC are subsequently induced to undergo maturation in the presence of the binding molecule.

In a further aspect of the invention, there is provided the use of a population of modulated DC obtained as a result of exposure to a CD45RO/RB binding molecule as described herein and/or a population of tolerogenic T-cells (i.e. Tr cells) obtained as a result of exposing T-cells to said tolerogenic DC, for the treatment and/or prophylaxis of disease associated with autoimmune disease, transplant rejection, psoriasis, inflammatory bowel disease and allergies.

In another aspect, the invention provides the use of a population of DC obtained as a result of exposure to a CD45RO/RB binding molecule as described herein and/or a population of tolerogenic T-cells (i.e. Tr cells) obtained as a result of exposing T-cells to said tolerogenic DC, for the manufacture of a medicament for the treatment and/or prophylaxis of disease associated with autoimmune disease, transplant rejection, psoriasis, inflammatory bowel disease and allergies.

The tolerogenic DC obtained as a result of exposure to a CD45RO/RB binding molecule as defined herein and/or the tolerogenic T-cells (i.e. Tr cells) obtained as a result of exposing T-cells to said tolerogenic DC find use as medicament and pharmaceutical compositions. In one embodiment, such medicaments/pharmaceutical compositions may additionally comprise a CD45RO/RB binding molecule as defined herein.

### DESCRIPTION OF THE FIGURES

FIG. 1. ChA6 mAb does not affect DC maturation. After 5 days of differentiation in IL-4 and GM-CSF, monocyte-derived DC were either left immature or matured for 48 h via activation of CD40L in the presence or absence of chA6 mAb (10 μg/ml). DC were then analyzed by flow cytometry to determine levels of expression of CD1a, CD14, CD83, HLA-DR, CD40, CD80 and CD86. Numbers indicate the percentages of positive cells. Results of one experiment representative of twenty independent experiments are shown.

FIG. 2. ChA6 mAb treatment modulates the expression of PDL-2 and CD45RB on mature DC. After 5 days of differentiation in IL-4 and GM-CSF, monocyte-derived DC were left either immature or matured for 48 h via activation of CD40L in the presence or absence of chA6 mAb (10 μg/ml). DC were then analyzed by flow cytometry to determine levels of the indicated markers. The average ±SEM amounts detected in the indicated independent experiments are presented. P values were calculated by T-test: *P comparison between mature/chA6 DC and mature DC and $P comparison between mature/chA6 DC and immature DC (*P or $P≦0.05, **P or $$P≦0.005).

FIG. 3. ChA6 mAb does not affect cytokine secretion by mature DC. After 5 days of differentiation in IL-4 and GM-CSF, monocyte-derived DC were matured for 48 h via activation of CD40 in the presence or absence of chA6 mAb (10 μg/ml). Mature (mDC) and chA6-modulated mature DC (chA6 mDC) were cultured, and supernatants were collected after 48 h. Levels of secreted IL-6, IL-10, TNF-α and IL-12 were determined by ELISA. The average ±SEM amounts detected in ten independent experiments are presented. No statistically differences were observed.

FIG. 4. ChA6-modulated mature DC induce hypo-responsive T-cells. Peripheral CD4+CD45RO- T-cells were repetitively activated with immature (Timm), mature (Tmat) or mature/chA6 (TchA6 mat) allogeneic DC for 3 rounds of stimulation. After the third round of stimulation, T-cell lines were tested for their ability to proliferate in response to allogeneic mDC (A). In addition, after the third round of activation, their proliferative response to polyclonal activation was tested by stimulation with immobilized anti-CD3 mAb (1 μg/ml), in the absence or presence of soluble anti-CD28 mAb (10 μg/ml) and IL-2 (100 U/ml) (B). After 48 hours of culture, [3H]-thymidine was added, for an additional 16 hours. Results are representative of 17 (A) and 3 (B) independent experiments.

FIG. 5. ChA6-modulated mature DC induce Tr cells. Peripheral CD4+CD45RO- T-cells were repetitively stimulated with immature (Timm), mature (Tmat) or mature/chA6 (TchA6 mat) allogeneic DC for 3 rounds of stimulation. After the third round of stimulation, T-cell lines were tested for their ability to proliferate in response to allogeneic mDC (open symbols) after 2, 3, and 4 days of culture, and for their ability to suppress responses of autologous CD4+ T-cells activated with mDC (closed symbols). Naïve CD4+ T-cells were stimulated with mature DCs alone (MLR) or in the presence of Timm, Tmat, and TchA6 maT-cell lines at a 1:1 ratio. [3H]-thymidine was added at the indicated time for an additional 16 h. Results of one experiment representative of 17 independent experiments are shown.

FIG. 6. Role of IL-10 and TGF-β in suppression mediated by Tr1 cells induced by chA6-modulated DC. After three rounds of activation with mature/chA6 DC, T(chA6 mat) cells were tested for their ability to suppress the proliferation of CD4+ T-cells in response to allogeneic monocytes, in the absence or presence of anti-IL-10R (30 μg/ml) and anti-TGF-β (50 μg/ml) mAbs. [3H]-thymidine was added at the indicated time for an additional 16 hours. Results are representative of 3 independent experiments.

FIG. 7. Signal through PDL-2 is required for the differentiation of Tr1 cells induced by chA6-modulated DC. Peripheral blood CD4+CD45RO- T-cells were stimulated with chA6-modulated allogeneic DC in the absence or presence of anti-PDL-2 or control IgG mAbs (10 μg/ml). After 3 rounds of stimulation, T-cells were collected and tested for their ability to proliferate in response to mature DC and to suppress the response of autologous CD4+ T-cells. [3H]-thymidine was added at the indicated time for an additional 16 hours. Results are representative of 3 independent experiments.

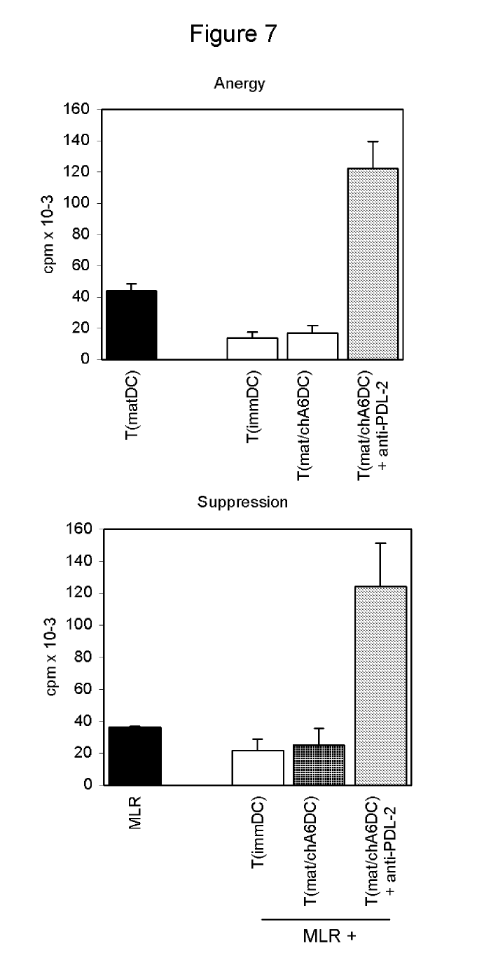

### DETAILED DESCRIPTION OF THE INVENTION

The invention is based on the appreciation that molecules which bind to the RO and RB isoforms of CD45 are capable of inducing a tolerogenic phenotype in dendritic cells. We have found that binding molecules which comprise a polypeptide sequence which binds to CD45RO and CD45RB, hereinafter also designated as “CD45RO/RB binding molecules” can induce tolerogenic dendritic cells which can function to inhibit primary T-cell responses and induce T-cell tolerance. It is demonstrated herein that anti-CD45RO/RB monoclonal antibodies do not prevent the maturation and activation of monocyte-derived dendritic cells, but do up-regulate the expression of PD-L2 and CD45RB on mature DC. By repetitive exposure of nave peripheral blood CD4⁺ T-cells to allogeneic DC, we have demonstrated that anti-CD45RO/RB monoclonal antibodies modulate DC function such that the DC induce differentiation of the peripheral blood CD4⁺ T-cells to a population of Tr cells which are phenotypically and functionally similar to Tr1 cells. Like Tr1 cells these Tr cells produce IL-10 and TGF-β and suppress T-cell responses via an IL-10- and TGF-β-dependent mechanism. In addition, we have demonstrated that signaling through PDL-2 is fundamental for Tr differentiation induced by the anti-CD45RO/RB modulated DC. In conclusion, it has been demonstrated that CD45RO/RB binding molecules function as immunomodulators through at least several modes of action, including deletion of effector T-cells and induction of Tr cells through modulation of dendritic cells.

By “CD45RO/RB binding molecule” it is meant any molecule capable of binding specifically to the CD45RB and CD45RO isoforms of the CD45 antigen, either alone or associated with other molecules. The binding reaction may be shown by standard methods (qualitative assay) including for example any kind of binding assay such as direct or indirect immunofluorescence together with fluorescence microscopy or cytofluorimetric (FACS) analysis, enzyme-linked immunosorbent assay (ELISA) or radioimmunoassay in which binding of the molecule to cells expressing a particular CD45 isoform can be visualized. In addition, the binding of this molecule may result in the alteration of the function of the cells expressing these isoforms, for example inhibition of primary or secondary mixed lymphocyte response (MLR) may be determined, such as an in vitro assay or a bioassay for determining the inhibition of primary or secondary MLR in the presence and in the absence of a CD45RO/RB binding molecule and determining the differences in primary MLR inhibition. An example of such an assay is as follows:

Human peripheral blood mononuclear cells (PBMC) or human CD3+ or CD4+ cells are mixed with irradiated allogeneic PBMC or T-cell-depleted irradiated (5000 rad) PBMC in each well of a 96-well culture plate in the presence of a CD45RO/RB binding molecule as defined herein, or in the presence of a control molecule such as mouse immunoglobulin-1. The cell mixture is cultured for 4 or 5 days at 37° C. in 5% CO2 and proliferation is determined by pulsing the cells with 3H-thymidine for the last 16-20 hours of culture. The percentage of inhibition of primary MLR is calculated in comparison with the cell proliferation in the presence of the control molecule. Secondary MLR inhibition may also be assessed.

Alternatively, in vitro functional modulatory effects can also be determined by measuring the PBMC or T-cells or CD4+ T-cells proliferation, production of cytokines, change in the expression of cell surface molecules e.g. following cell activation in MLR, or following stimulation with specific antigen such as tetanus toxoid or other antigens, or with polyclonal stimulators such as phytohemagglutinin (PHA) or anti-CD3 and anti-CD28 antibodies or phorbol esters and Ca++ ionophores. The cultures are set up in a similar manner as described for MLR except that instead of allogeneic cells as stimulators soluble antigen or polyclonal stimulators such as those mentioned above are used. T-cell proliferation is measured preferably as described above by 3H-thymidine incorporation. Cytokine production is measured by sandwich ELISA where a cytokine capture antibody is coated on the surface of a 96-well tray, the supernatants from the cultures are added and incubated for 1 hr at room temperature and a detecting antibody specific for the particular cytokine is then added, following a second-step antibody conjugated to an enzyme such as Horseradish peroxidase followed by the corresponding substrate and the absorbance is measured in a plate reader. The change in cell surface molecules is measured by direct or indirect immunofluorescence after staining the targeT-cells with antibodies specific for a particular cell surface molecule. The antibody can be either directly labeled with flourochrome or a fluorescently labeled second step antibody specific for the first antibody can be used, and the cells are analysed with a cytofluorimeter.

The binding molecule used in the invention has a binding specificity for both CD45RO and CD45RB (“CD45 RB/RO binding molecule”).

Preferably the binding molecule binds to CD45RO isoforms with a dissociation constant (Kd)<20 nM, preferably with a Kd<15 nM or <10 nM, or preferably with a Kd<5 nM. Preferably the binding molecule binds to CD45RB isoforms with a Kd<50 nM, preferably with a Kd<15 nM or <10 nM, more preferably with a Kd<5 nM.

In a further preferred embodiment the binding molecule utilized in the present invention binds those CD45 isoforms which

1) include the A and B epitopes but not the C epitope of the CD45 molecule; and/or
2) include the B epitope but not the A and not the C epitope of the CD45 molecule; and/or
3) do not include any of the A, B or C epitopes of the CD45 molecule.

In yet a further preferred embodiment the binding molecule does not bind CD45 isoforms which include

1) all of the A, B and C epitopes of the CD45 molecule; and/or
2) both the B and C epitopes but not the A epitope of the CD45 molecule.

In further preferred embodiments the binding molecule

1) recognises memory and in vivo alloactivated T-cells; and/or
2) binds to its target on human T-cells, such as for example PEER cells; wherein said binding preferably is with a Kd<15 nM, more preferably with a Kd<10 nM, most preferably with a Kd<5 nM; and/or
3) inhibits in vitro alloreactive T-cell function, preferably with an IC50 of about less than 100 nM, preferably less than 50 nM or 30 nM, more preferably with an IC50 of about 10 or 5 nM, most preferably with an IC50 of about 0,5 nM or even 0,1 nM; and/or
4) induces cell death through apoptosis in human T lymphocytes; and/or
5) induces alloantigen-specific T-cell tolerance in vitro; and/or
6) prevents lethal xenogeneic graft versus host disease (GvHD) induced in SCID mice by injection of human PBMC when administered in an effective amount; and/or
7) binds to T lymphocytes, monocytes, stem cells, natural killer cells and/or granulocytes, but not to platelets or B lymphocytes; and/or
8) supports the differentiation of T-cells with a characteristic T regulatory cell (Treg) phenotype; and/or
9) induces T regulatory cells capable of suppressing nave T-cell activation; and/or
10) suppresses the inflammatory process that mediates human allograft skin rejection, in particular, suppresses the inflammatory process that mediates human allograft skin rejection in vivo in SCID mice transplanted with human skin and engrafted with mononuclear splenocytes; and/or
11) prolongs human islet allograft survival in a hu-PBL-NOD/SCID mice model.

In a further preferred embodiment the binding molecule used in the present invention binds to the same epitope as the monoclonal antibody “A6” as described by Aversa et al., Cellular Immunology 158, 314-328 (1994). The entire contents of this reference is incorporated herein by reference and to which the reader is specifically referred.

Due to the above-described binding properties and biological activities, the binding molecules made use of in the present invention are particularly useful in medicine, for therapy and/or prophylaxis. In addition, such binding molecules are particularly useful in modulating DC function ex vivo such that the DC exhibits a tolerogenic phenotype. It is envisaged that these tolerogenic DC will be useful in therapy and/or prophylaxis. Diseases in which binding molecules and/or the modulated DC are particularly useful include autoimmune diseases, transplant rejection, dermatitis, psoriasis, inflammatory bowel disease and/or allergies, as will be further set out below.

A molecule comprising a polypeptide of SEQ ID NO: 1 and a polypeptide of SEQ ID NO: 2 is a CD45RO/RB binding molecule. The hypervariable regions CDR1′, CDR2′ and CDR3′ in the CD45RO/RB binding molecule of SEQ ID NO:1 is the following; CDR1′ having the amino acid sequence Arg-Ala-Ser-Gln-Asn-Ile-Gly-Thr-Ser-Ile-Gln (RASQNIGTSIQ) (SEQ ID NO:19), CDR2′ having the amino acid sequence Ser-Ser-Ser-Glu-Ser-Ile-Ser (SSSESIS) (SEQ ID NO:20) and CDR3′ having the amino acid sequence Gln-Gln-Ser-Asn-Thr-Trp-Pro-Phe-Thr (QQSNTWPFT) (SEQ ID NO:21).

We also have found the hypervariable regions CDR1, CDR2 and CDR3 in a CD45RO/RB binding molecule of SEQ ID NO:2, CDR1 having the amino acid sequence Asn-Tyr-Ile-Ile-His (NYIIH) (SEQ ID NO:22), CDR2 having the amino acid sequence Tyr-Phe-Asn-Pro-Tyr-Asn-His-Gly-Thr-Lys-Tyr-Asn-Glu-Lys-Phe-Lys-Gly (YFNPYNHGTKYNEKFKG) (SEQ ID NO:23) and CDR3 having the amino acid sequence Ser-Gly-Pro-Tyr-Ala-Trp-Phe-Asp-Thr (SGPYAWFDT) (SEQ ID NO:24).

CDRs are 3 specific complementary determining regions which are also called hypervariable regions which essentially determine the antigen binding characteristics. These CDRs are part of the variable region, e.g. of SEQ ID NO: 1 or SEQ ID NO: 2, respectively, wherein these CDRs alternate with framework regions (FR's) e.g. constant regions. A SEQ ID NO: 1 is part of a light chain, e.g. of SEQ ID NO: 3, and a SEQ ID NO:2 is part of a heavy chain, e.g. of SEQ ID NO: 4, in a chimeric antibody. The CDRs of a heavy chain together with the CDRs of an associated light chain essentially constitute the antigen binding site of a molecule utilized by the present invention. It is known that the contribution made by a light chain variable region to the energetics of binding is small compared to that made by the associated heavy chain variable region and that isolated heavy chain variable regions have an antigen binding activity on their own. Such molecules are commonly referred to as single domain antibodies.

In one embodiment of the present invention the binding molecule utilized comprises at least one antigen binding site, e.g. a CD45RO/RB binding molecule, comprising in sequence the hypervariable regions CDR1, CDR2 and CDR3, said CDR1 having the amino acid sequence Asn-Tyr-Ile-Ile-His (NYIIH) (SEQ ID NO:22), said CDR2 having the amino acid sequence Tyr-Phe-Asn-Pro-Tyr-Asn-His-Gly-Thr-Lys-Tyr-Asn-Glu-Lys-Phe-Lys-Gly (YFNPYNHGTKYNEKFKG) (SEQ ID NO:23) and said CDR3 having the amino acid sequence Ser-Gly-Pro-Tyr-Ala-Trp-Phe-Asp-Thr (SGPYAWFDT) (SEQ ID NO:24). In yet a further embodiment, the binding molecule is a direct equivalent of the binding molecule structurally defined above.

In another aspect the present invention makes use of a molecule comprising at least one antigen binding site, e.g. a CD45RO/RB binding molecule, comprising

a) a first domain comprising in sequence the hypervariable regions CDR1, CDR2 and CDR3, said CDR1 having the amino acid sequence Asn-Tyr-Ile-Ile-His (NYIIH) (SEQ ID NO:22), said CDR2 having the amino acid sequence Tyr-Phe-Asn-Pro-Tyr-Asn-His-Gly-Thr-Lys-Tyr-Asn-Glu-Lys-Phe-Lys-Gly (YFNPYNHGTKYNEKFKG) (SEQ ID NO:23) and said CDR3 having the amino acid sequence Ser-Gly-Pro-Tyr-Ala-Trp-Phe-Asp-Thr (SGPYAWFDT) (SEQ ID NO:24); and
b) a second domain comprising in sequence the hypervariable regions CDR1′, CDR2′ and CDR3′, CDR1′ having the amino acid sequence Arg-Ala-Ser-Gln-Asn-Ile-Gly-Thr-Ser-Ile-Gln (RASQNIGTSIQ) (SEQ ID NO:19), CDR2′ having the amino acid sequence Ser-Ser-Ser-Glu-Ser-Ile-Ser (SSSESIS) (SEQ ID NO:20) and CDR3′ having the amino acid sequence Gln-Gln-Ser-Asn-Thr-Trp-Pro-Phe-Thr (QQSNTWPFT) (SEQ ID NO:21). In an alternative embodiment, the invention makes use of a binding molecule which is a direct equivalent of the binding molecule described directly above.

In a preferred embodiment the first domain comprising in sequence the hypervariable regions CDR1, CDR2 and CDR3 is an immunoglobulin heavy chain, and the second domain comprising in sequence the hypervariable regions CDR1′, CDR2′ and CDR3′ is an immunoglobulin light chain.

In a further aspect the present invention makes use of a molecule, e.g. a CD45RO/RB binding molecule, comprising a polypeptide of SEQ ID NO: 1 and/or a polypeptide of SEQ ID NO: 2, preferably comprising in one domain a polypeptide of SEQ ID NO: 1 and in another domain a polypeptide of SEQ ID NO: 2, e.g. a chimeric monoclonal antibody. In another aspect the invention makes use of a molecule, e.g. a CD45RO/RB binding molecule, comprising a polypeptide of SEQ ID NO: 3 and/or a polypeptide of SEQ ID NO: 4, preferably comprising in one domain a polypeptide of SEQ ID NO: 3 and in another domain a polypeptide of SEQ ID NO: 4, e.g. a chimeric monoclonal antibody. When the antigen binding site comprises both the first and second domains or a polypeptide of SEQ ID NO: 1 or SEQ ID NO:3, respectively, and a polypeptide of SEQ ID NO: 2 or of SEQ ID NO:4, respectively, these may be located on the same polypeptide, or, preferably each domain may be on a different chain, e.g. the first domain being part of an heavy chain, e.g. immunoglobulin heavy chain, or fragment thereof and the second domain being part of a light chain, e.g. an immunoglobulin light chain or fragment thereof.

As can be seen from the description given above, in preferred embodiments the CD45RO/RB binding molecule utilized according to the present invention is a monoclonal antibody (mAb), wherein the binding activity is determined mainly by the CDR regions as described above, e.g. said CDR regions being associated with other molecules without binding specificity, such as framework, e.g. constant regions, which are substantially of human origin. In a preferred embodiment, the CD45RO/RB binding molecule is a monoclonal antibody of the IgG1 isotype.

The present invention may utilize a CD45RO/RB binding molecule which is the monoclonal antibody “A6” as described by Aversa et al., Cellular Immunology 158, 314-328 (1994), which is incorporated by reference for the passages characterizing A6.

In another aspect the present invention utilizes a CD45RO/RB binding molecule according to the present invention which is a chimeric, a humanised or a fully human monoclonal antibody.

Examples of CD45RO/RB binding molecules include chimeric or humanised antibodies e.g. derived from antibodies as produced by B-cells or hybridomas and/or any fragment thereof, e.g. F(ab′)2 and Fab fragments, as well as single chain or single domain antibodies. A single chain antibody consists of the variable regions of antibody heavy and light chains covalently bound by a peptide linker, usually consisting of from 10 to 30 amino acids, preferably from 15 to 25 amino acids. Therefore, such a structure does not include the constant part of the heavy and light chains and it is believed that the small peptide spacer should be less antigenic than a whole constant part. By a chimeric antibody is meant an antibody in which the constant regions of heavy and light chains or both are of human origin while the variable domains of both heavy and light chains are of non-human (e.g. murine) origin. By a humanised antibody is meant an antibody in which the hypervariable regions (CDRs) are of non-human (e.g. murine) origin while all or substantially all the other part, e.g. the constant regions and the highly conserved parts of the variable regions are of human origins. A humanised antibody may however retain a few amino acids of the murine sequence in the parts of the variable regions adjacent to the hypervariable regions.

Hypervariable regions, i.e. CDR's may be associated with any kind of framework regions, e.g. constant parts of the light and heavy chains, of human origin. Suitable framework regions are e.g. described in “Sequences of proteins of immunological interest”, Kabat, E. A. et al, US department of health and human services, Public health service, National Institute of health. Preferably the constant part of a human heavy chain is of the IgG1 type, including subtypes, preferably the constant part of a human light chain may be of the κ or λ type, more preferably of the κ type. Preferably, said heavy chain comprises not more than one glycosylation site, most preferably the glycosylation site is an N-glycosylation site, and most preferably the one glycosylation site is located in the constant part of the heavy chain. Most preferably no glycosylation site is present in the variable region, preferably no glycosylation site in the framework region.

A preferred constant part of a heavy chain is a polypeptide of SEQ ID NO: 4 (without the CDR1′, CDR2′ and CDR3′ sequence parts which are specified above) and a preferred constant part of a light chain is a polypeptide of SEQ ID NO: 3 (without the CDR1, CDR2 and CDR3 sequence parts which are specified above).

In one embodiment, a humanised antibody is utilized comprising a light chain variable region of amino acid SEQ ID NO:7 or of amino acid SEQ ID NO:8, which comprises CDR1′, CDR2′ and CDR3′ as defined above and/or a heavy chain variable region of SEQ:ID NO:9 or of SEQ:ID NO:10, which comprises CDR1, CDR2 and CDR3 as defined above.

In a further embodiment, another humanised antibody is utilized comprising a light chain variable region of amino acid SEQ ID NO:7 or of amino acid SEQ ID NO:8, which comprises CDR1′, CDR2′ and CDR3′ as defined above and/or a heavy chain variable region of SEQ:ID NO:31 or of SEQ:ID NO:32, which comprises CDR1, CDR2 and CDR3 as defined above.

In yet another embodiment, the present invention makes use of a humanised antibody comprising a polypeptide of SEQ ID NO:9 or of SEQ ID NO:10 and a polypeptide of SEQ ID NO:7 or of SEQ ID NO:8. In a still further embodiment, the invention uses a humanised antibody comprising a polypeptide of SEQ ID NO:31 or of SEQ ID NO:32 and a polypeptide of SEQ ID NO:7 or of SEQ ID NO:8.

In further embodiments the present invention makes use of a humanised antibody comprising
a polypeptide of SEQ ID NO:9 and a polypeptide of SEQ ID NO:7 (such as VHE/humV2),
a polypeptide of SEQ ID NO:9 and a polypeptide of SEQ ID NO:8 (such as VHE/humV1),
a polypeptide of SEQ ID NO:10 and a polypeptide of SEQ ID NO:7 (such as VHQ/humV2),
a polypeptide of SEQ ID NO:10 and a polypeptide of SEQ ID NO:8 (such as VHQ/humV1),
a polypeptide of SEQ ID NO:31 and a polypeptide of SEQ ID NO:7 (such as VHEN73D/humV2),
a polypeptide of SEQ ID NO:31 and a polypeptide of SEQ ID NO:8 (such as VHEN73D/humV1),
a polypeptide of SEQ ID NO:32 and a polypeptide of SEQ ID NO:7 (such as VHQN73D/humV2), or
a polypeptide of SEQ ID NO:32 and a polypeptide of SEQ ID NO:8 (such as VHQN73D/humV1).

Reference to a polypeptide utilized according to the present invention, e.g. of a herein specified sequence, e.g. of CDR1 (SEQ ID NO:22), CDR2 (SEQ ID NO:23), CDR3 (SEQ ID NO:24), CDR1′ (SEQ ID NO:19), CDR2′ (SEQ ID NO:20), CDR3′ (SEQ ID NO:21), or of a SEQ ID NO:1, SEQ ID NO:2, SEQ ID NO:3, SEQ ID NO:4, SEQ ID NO:7, SEQ ID NO:8, SEQ ID NO:9, SEQ ID NO:10, SEQ ID NO:31 or SEQ ID NO:32 includes direct equivalents of said (poly)peptide (sequence); e.g. including a functional derivative of said polypeptide. Said functional derivative may include covalent modifications of a specified sequence, and/or said functional derivative may include amino acid sequence variants of a specified sequence.

“Polypeptide”, if not otherwise specified herein, includes any peptide or protein comprising amino acids joined to each other by peptide bonds, having an amino acid sequence starting at the N-terminal extremity and ending at the C-terminal extremity. Preferably polypeptides made us of in the present invention are monoclonal antibodies. More preferably the polypeptides are chimeric (V-grafted) or humanised (CDR-grafted) monoclonal antibodies. The humanised (CDR-grafted) monoclonal antibody may or may not include further mutations introduced into the framework (FR) sequences of the acceptor antibody. Preferably the humanized or chimeric antibody comprises no more than one glycosylation site. Most preferably said one glycosylation site is a N-glycosylation site. Most preferably no glycosylation site is present in the variable region, and even more preferably no glycosylation site is present in the variable region of the heavy chain, most preferably no glycosylation site is present in the framework regions (FR's).

A functional derivative of a polypeptide as used herein includes a molecule having a qualitative biological activity in common with a polypeptide used in the present invention, i.e. having the ability to bind to CD45RO and CD45RB. A functional derivative includes fragments and peptide analogs of a polypeptide utilized according to the present invention. Fragments comprise regions within the sequence of a polypeptide, e.g. of a specified sequence. The term “derivative” is used to define amino acid sequence variants, and covalent modifications of a polypeptide made use of in the present invention. e.g. of a specified sequence. The functional derivatives of a polypeptide utilized according to the present invention, e.g. of a specified sequence, preferably have at least about 65%, more preferably at least about 75%, even more preferably at least about 85%, most preferably at least about 95% overall sequence homology with the amino acid sequence of a polypeptide as structurally defined above, e.g. of a specified sequence, and substantially retain the ability to bind to CD45RO and CD45RB.

Preferably, the functional derivative has at least the binding affinity of a binding molecule comprising a polypeptide of SEQ ID NO:1 and/or a polypeptide of SEQ ID NO:2, of a humanised antibody comprising a polypeptide of SEQ ID NO:9 or of SEQ ID NO:10 and/or a polypeptide of SEQ ID NO:7 or of SEQ ID NO:8; or of a humanised antibody comprising a polypeptide of SEQ ID NO:31 or of SEQ ID NO:32 and/or a polypeptide of SEQ ID NO:7 or of SEQ ID NO:8.

The term “covalent modification” includes modifications of a polypeptide as defined herein, e.g. of a specified sequence; or a fragment thereof with an organic proteinaceous or non-proteinaceous derivatizing agent, fusions to heterologous polypeptide sequences, and post-translational modifications. Covalent modified polypeptides, e.g. of a specified sequence, still have the ability bind to CD45RO and CD45RB by crosslinking Covalent modifications are traditionally introduced by reacting targeted amino acid residues with an organic derivatizing agent that is capable of reacting with selected sides or terminal residues, or by harnessing mechanisms of post-translational modifications that function in selected recombinant hosT-cells. Certain post-translational modifications are the result of the action of recombinant hosT-cells on the expressed polypeptide. Glutaminyl and asparaginyl residues are frequently post-translationally deaminated to the corresponding glutamyl and aspartyl residues. Alternatively, these residues are deaminated under mildly acidic conditions. Other post-translational modifications include hydroxylation of proline and lysine, phosphorylation of hydroxyl groups of seryl, tyrosine or threonyl residues, methylation of the α-amino groups of lysine, arginine, and histidine side chains, see e.g. T. E. Creighton, Proteins: Structure and Molecular Properties, W.H. Freeman & Co., San Francisco, pp. 79-86 (1983). Covalent modifications e.g. include fusion proteins comprising a polypeptide as defined herein, e.g. of a specified sequence and their amino acid sequence variants, such as immunoadhesins, and N-terminal fusions to heterologous signal sequences.

“Homology” with respect to a native polypeptide and its functional derivative is defined herein as the percentage of amino acid residues in the candidate sequence that are identical with the residues of a corresponding native polypeptide, after aligning the sequences and introducing gaps, if necessary, to achieve the maximum percent homology, and not considering any conservative substitutions as part of the sequence identity. Neither N- nor C-terminal extensions nor insertions shall be construed as reducing identity or homology. Methods and computer programs for the alignment are well known.

“Amino acid(s)” refer to all naturally occurring L-a-amino acids, e.g. and including D-amino acids. The amino acids are identified by either the well known single-letter or three-letter designations.

The term “amino acid sequence variant” refers to molecules with some differences in their amino acid sequences as compared to a polypeptide as defined herein, e.g. of a specified sequence. Amino acid sequence variants of a polypeptide as defined herein, e.g. of a specified sequence, still have the ability to bind to CD45RO and CD45RB.

Substitutional variants are those that have at least one amino acid residue removed and a different amino acid inserted in its place at the same position in a polypeptide as defined herein, e.g. of a specified sequence. These substitutions may be single, where only one amino acid in the molecule has been substituted, or they may be multiple, where two or more amino acids have been substituted in the same molecule. Insertional variants are those with one or more amino acids inserted immediately adjacent to an amino acid at a particular position in a polypeptide as defined herein, e.g. of a specified sequence. Immediately adjacent to an amino acid means connected to either the α-carboxy or α-amino functional group of the amino acid. Deletional variants are those with one or more amino acids in a polypeptide according to the present invention, e.g. of a specified sequence, removed. Ordinarily, deletional variants will have one or two amino acids deleted in a particular region of the molecule.

Also described herein are the polynucleotide sequences of:
GGCCAGTCAGAACATTGGCACAAGCATACAGTG (SEQ ID NO:25), encoding the amino acid sequence of CDR1;
TTCTTCTGAGTCTATCTCTGG (SEQ ID NO:26), encoding the amino acid sequence of CDR 2;
ACAAAGTAATACCTGGCCATTCACGTT (SEQ ID NO:27), encoding the amino acid sequence of CDR 3;
TTATATTATCCACTG (SEQ ID NO:28), encoding the amino acid sequence of CDR1′,
TTTTAATCCTTACAATCATGGTACTAAGTACAATGAGAAGTTCAAAGGCAG (SEQ ID NO:29), encoding the amino acid sequence of CDR2;
AGGACCCTATGCCTGGTTTGACACCTG (SEQ ID NO:30), encoding the amino acid sequence of CDR3;
SEQ ID NO:5 encoding a polypeptide of SEQ ID NO: 1, i.e. the variable region of a light chain of an mAb utilized according to the present invention;
SEQ ID NO:6 encoding a polypeptide of SEQ ID NO:2, i.e. the variable region of the heavy chain of an mAb utilized according to the present invention;
SEQ ID NO:11 encoding a polypeptide of SEQ ID NO:9. i.e. a heavy chain variable region including CDR1, CDR2 and CDR3;
SEQ ID NO:12 encoding a polypeptide of SEQ ID NO:10, i.e. a heavy chain variable region including CDR1, CDR2 and CDR3;
SEQ ID NO:13 encoding a polypeptide of SEQ ID NO:7, i.e. a light chain variable region including CDR1′, CDR2′ and CDR3;
SEQ ID NO:14 encoding a polypeptide of SEQ ID NO:8, i.e. a light chain variable region including CDR1′, CDR2′ and CDR3;
SEQ ID NO:33 encoding a polypeptide of SEQ ID NO:8, i.e. a light chain variable region including CDR1′, CDR2′ and CDR3;
SEQ ID NO:34 encoding a polypeptide of SEQ ID NO:31, i.e. a heavy chain variable region including CDR1, CDR2 and CDR3; and
SEQ ID NO:35 encoding a polypeptide of SEQ ID NO:32, i.e. a heavy chain variable region including CDR1, CDR2 and CDR3;

Polynucleotides comprising polynucleotides encoding a CD45RO/RB binding molecule, e.g. encoding the amino acid sequence of CDR1, CDR2 and CDR3 as defined herein and/or polynucleotides encoding the amino acid sequence of CDR1′, CDR2′ and CDR3′ as defined herein can be used as a source material for the generation of the binding molecules made use of by the present invention. Such polynucleotides include those listed above as well as those set out below, as follows:

Polynucleotides comprising a polynucleotide of SEQ ID NO: 5 and/or, preferably and, a polynucleotide of SEQ ID NO: 6;

Polynucleotides comprising polynucleotides encoding a polypeptide of SEQ ID NO:7 or SEQ ID NO:8 and/or, preferably and, a polypeptide of SEQ ID NO:9 or SEQ ID NO:10; e.g. encoding
a polypeptide of SEQ ID NO:7 and a polypeptide of SEQ ID NO:9,
a polypeptide of SEQ ID NO:7 and a polypeptide of SEQ ID NO:10,
a polypeptide of SEQ ID NO:8 and a polypeptide of SEQ ID NO:9, or
a polypeptide of SEQ ID NO:8 and a polypeptide of SEQ ID NO:10;

Polynucleotides comprising a polynucleotide of SEQ ID NO:11 or of SEQ ID NO:12 and/or, preferably and, a polynucleotide of SEQ ID NO:13 or a polynucleotide of SEQ ID NO:14, preferably comprising
a polynucleotide of SEQ ID NO:11 and a polynucleotide of SEQ ID NO:13,
a polynucleotide of SEQ ID NO:11 and a polynucleotide of SEQ ID NO:14,
a polynucleotide of SEQ ID NO:12 and a polynucleotide of SEQ ID NO:13, or
a polynucleotide of SEQ ID NO:12 and a polynucleotide of SEQ ID NO:14;

Polynucleotides comprising polynucleotides encoding a polypeptide of SEQ ID NO:31 or of SEQ ID NO:32 and/or, preferably and, a polypeptide of SEQ ID NO:7 or of SEQ ID NO:8; e.g. encoding
a polypeptide of SEQ ID NO:31 and a polypeptide of SEQ ID NO:7,
a polypeptide of SEQ ID NO:31 and a polypeptide of SEQ ID NO:8,
a polypeptide of SEQ ID NO:32 and a polypeptide of SEQ ID NO:7, or
a polypeptide of SEQ ID NO:32 and a polypeptide of SEQ ID NO:8; and

Polynucleotides comprising a polynucleotide of SEQ ID NO:34 or of SEQ ID NO:35 and/or, preferably and, a polynucleotide of SEQ ID NO:33; SEQ ID NO:14 or 13.
a polypeptide of SEQ ID NO:34 and a polypeptide of SEQ ID NO:33,
a polypeptide of SEQ ID NO:34 and a polypeptide of SEQ ID NO:14,
a polypeptide of SEQ ID NO:34 and a polypeptide of SEQ ID NO:13,
a polypeptide of SEQ ID NO:35 and a polypeptide of SEQ ID NO:33,
a polypeptide of SEQ ID NO:35 and a polypeptide of SEQ ID NO:14, or
a polypeptide of SEQ ID NO:35 and a polypeptide of SEQ ID NO:13.

“Polynucleotide”, if not otherwise specified herein, includes any polyribonucleotide or polydeoxyribonucleotide, which may be unmodified RNA or DNA, or modified RNA or DNA, including without limitation single and double stranded RNA, and RNA that is a mixture of single- and double-stranded regions.

A CD45RO/RB binding molecule, e.g. which is a chimeric, humanised or fully human antibody, may be produced by recombinant DNA techniques. Thus, one or more DNA molecules encoding the CD45RO/RB may be constructed, placed under appropriate control sequences and transferred (e.g. by transfection) into a suitable host (organism) for expression by an appropriate vector.

Such polynucleotides may for example encode a single, heavy and/or a light chain of a CD45RO/RB binding molecule.

A CD45RO/RB binding molecule may be obtained by conventional methods together with the information provided herein, e.g. with the knowledge of the amino acid sequence of the hypervariable or variable regions and the polynucleotide sequences encoding these regions. A method for constructing a variable domain gene is e.g. described in EP 239 400 and may be briefly summarized as follows: A gene encoding a variable region of a mAb of whatever specificity may be cloned. The DNA segments encoding the framework and hypervariable regions are determined and the DNA segments encoding the hypervariable regions are removed. Double stranded synthetic CDR cassettes are prepared by DNA synthesis according to the CDR and CDR' sequences as specified herein. These cassettes are provided with sticky ends so that they can be ligated at junctions of a desired framework of human origin. Polynucleotides encoding single chain antibodies may also be prepared according to, e.g. analogously, to a method as conventional. A polynucleotide encoding a polypeptide used in the present invention may be conveniently transferred into an appropriate expression vector.

Appropriate cell lines (such as CHO cell lines, e.g. DG44 and other DHFR⁻ CHO cell, Sp/2 or NS/0 cell lines) may be used according to conventional methods. Expression vectors, e.g. comprising suitable promotor(s) and genes encoding heavy and light chain constant parts are known e.g. and are commercially available. Appropriate hosts (including cell cultures or transgenic animals) are known or may be found according to conventional methods.

Suitable expression vectors include a polynucleotide encoding a CD45RO/RB binding molecule as defined herein, e.g. of sequence SEQ ID NO:15, SEQ ID NO:16, SEQ ID NO:17, SEQ ID NO:18, SEQ ID NO:36, SEQ ID NO:37, SEQ ID NO:38, SEQ ID NO:39, SEQ ID NO:40 or SEQ ID NO:41.

As described above, a CD45RO/RB binding molecule used according to the present invention exerts an immunosuppressive and tolerogenic effect through the modulation of DC phenotype. These previously unappreciated properties exhibited by CD45RO/RB binding molecules make them useful for both in vivo and ex-vivo tolerance induction to alloantigens, autoantigens, allergens and bacterial flora antigens. For example, CD45RO/RB binding molecules may be useful for the ex vivo induction of tolerogenic DC which can, following exposure to the binding molecules, be introduced into a host in need thereof, for the treatment and prophylaxis of diseases e.g. including autoimmune diseases, such as, but not limited to, rheumatoid arthritis, psoriatic arthritis, autoimmune thyroditis, Graves disease, type I and type II diabetes, multiple sclerosis, Crohn's disease (CD), ulcerative colitis (UC), systemic lupus erythematosus, Sjögren syndrome, scleroderma, autoimmune gastritis, glomerulonephritis, transplant rejection, such as, but not limited to, organ and tissue allograft and xenograft rejection, e.g. for the treatment of recipients of e.g. heart, lung, combined heart-lung, liver, kidney, pancreatic, skin or corneal transplants, graft versus host disease (GVHD), such as following bone marrow transplantation, and/or pancreatic isleT-cell transplant rejection, and/or also psoriasis, dermatitis such as atopic and contact dermatitis including allergic contact dermatitis, inflammatory bowel disease and/or allergies, including allergic asthma. In preferred embodiments, the method and compositions of the invention concern the treatment and/or prophylaxis of psoriasis and transplant rejection (for example in ameliorating rejection by a human recipient of transplanted allogeneic cells such as pancreatic islet cells).

It is envisaged that DC modulated by exposure to a CD45RO/RB binding molecule as defined herein, will be useful pharmaceuticals/medicaments, e.g. for the treatment and/or prophylaxis of autoimmune diseases, transplant rejection, e.g. pancreatic isleT-cell transplant rejection or graft versus host disease (GVHD), psoriasis, dermatitis, inflammatory bowel disease and/or allergies.

An “effective amount” of DC and/or Tr cells, as used herein, is an amount sufficient to bring about beneficial or desired results including clinical results such as decreasing one or more symptoms resulting from the autoimmune disease, transplant rejection, psoriasis, dermatitis, inflammatory bowel disease and/or allergy, increasing the quality of life of those suffering from, decreasing the dose of other medications required to treat such diseases, enhancing effect of another medication, delaying the progression of the disease, and/or prolonging survival of patients, either directly or indirectly.

An effective amount can be administered in one or more administrations and may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

The DC modulated according to the present invention and/or Tr resulting from exposure of T-cells to modulated DC, may be administered as the sole active ingredient(s) or together with other drugs in immunomodulating regimens or other anti-inflammatory agents e.g. for the treatment or prevention of diseases associated with autoimmune diseases, transplant rejection, psoriasis, dermatitis inflammatory bowel disease and/or allergies. For example, the DC and/or Tr may be used in combination with a calcineurin inhibitor, e.g. cyclosporine A, cyclosporine G, FK-506, ABT-281, ASM 981; an mTOR inhibitor, e.g. rapamycin, 40-O-(2-hydroxy)ethyl-rapamycin, CCI779, ABT578, AP23573, AP23464, AP23675, AP23841, TAFA-93, biolimus-7 or bioimus-9; a corticosteroid; cyclophosphamide; azathioprine; methotrexate; a SIP receptor agonist, e.g. FTY 720 or an analogue thereof; leflunomide or analogs thereof; mizoribine; mycophenolic acid; mycophenolate mofetil; 15-deoxyspergualine or analogs thereof; immunosuppressive monoclonal antibodies, e.g., monoclonal antibodies to leukocyte receptors, e.g., MHC, CD2, CDS, CD4, CD11a/CD18, CD7, CD25, CD27, B7, CD40, CD45, CD58, CD137, ICOS, CD150 (SLAM), OX40, 4-1BB or their ligands, e.g. CD154; or other immunomodulatory compounds, e.g. a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA41g (e.g. designated ATCC 68629) or a mutant thereof, e.g. LEA29Y, or other adhesion molecule inhibitors, e.g. mAbs or low molecular weight inhibitors including LFA-1 antagonists, Selectin antagonists and VLA-4 antagonists.

Administration can be by any conventional route, including injection or by gradual infusion over time. The administration may, for example, be intravenous, intraperitoneal, intramuscular, intracavity, subcutaneous, topical or transdermal. By “co administration” is meant administration of the components together or at substantially the same time, either in the same vehicle or in separate vehicles

Preferably, components are administered as a fixed combination.

The medicaments and pharmaceutical compositions of the invention may include at least one pharmaceutically acceptable carrier or diluent.

The term “pharmaceutically-acceptable carrier or diluent” as used herein means one or more compatible solid or liquid fillers, diluents or encapsulating substances which are suitable for administration into a mammals including humans. The term “carrier” denotes an organic or inorganic ingredient, natural or synthetic, with which the active ingredient is combined to facilitate the application.

The term “pharmaceutically acceptable” means a non-toxic material that does not interfere with the effectiveness of the biological activity of the active ingredients. Such preparations may routinely contain pharmaceutically acceptable concentrations of salts, buffering agents, preservatives, compatible carriers, supplementary immune potentiating agents such as adjuvants and cytokines and optionally other therapeutic agents, such as chemotherapeutic agents.

When used in medicine, the salts should be pharmaceutically acceptable, but non-pharmaceutically acceptable salts may conveniently be used to prepare pharmaceutically-acceptable salts thereof.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

The doses of DC and/or Tr cells administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

The pharmaceutical compositions/medicaments of the invention may comprise further, e.g. active, ingredients, e.g. other immunomodulatory antibodies such as, but not confined to a CD45RO/RB binding molecule as defined herein, anti-ICOS, anti-CD154, anti-CD134L or recombinant proteins such as, but not confined to rCTLA-4 (CD152), rOX40 (CD134), or anti-inflammatory agents or immunomodulatory compounds such as, but not confined to cyclosporin A, FTY720, RAD, rapamycin, FK506, 15-deoxyspergualin, steroids; as described above. Such

The compositions of the invention can be administered as a free combination, or can be formulated into a fixed combination. Absolute dosages will vary depending on a number of factors, e.g. the individual, the route of administration, the desired duration, the rate of release of the active agent and the nature and severity of the condition to be treated. Diseases as outlined above to be treated according to the methods and uses of the present invention include, but are not limited to autoimmune diseases, including rheumatoid arthritis, psoriatic arthritis, autoimmune thyroditis, Graves disease, type I and type II diabetes, multiple sclerosis, Crohn's disease (CD), ulcerative colitis (UC), systemic lupus erythematosus, Sjögren syndrome, scleroderma, autoimmune gastritis and glomerulonephritis; transplant rejection, including, but are not limited to, organ and tissue allograft and xenograft rejection, e.g. for the treatment of recipients of e.g. heart, lung, combined heart-lung, liver, kidney, pancreatic, skin or corneal transplants, graft versus host disease (GVHD), such as following bone marrow transplantation, and/or pancreatic isleT-cell transplant rejection; psoriasis; dermatitis such as atopic and contact dermatitis including allergic contact dermatitis; inflammatory bowel disease and/or allergies, including allergic asthma.

### EXAMPLES

The invention will become more fully understood by reference to the following examples. These examples are for illustrative purposes only and should not be construed as limiting the scope of the invention.

The antibody referred to herein as chA6 mAb is a chimeric antibody comprising a light chain of SEQ ID NO:3 and a heavy chain of SEQ ID NO:4.

All analysis for statistically significant differences was performed with the student's paired t test. p values less than 0.05 were considered significant. All cultures were performed in triplicate and error bars represent the SD.

### Example 1

### Generation of chimeric A6 antibody (chA6)

ChA6 was generated by linking the variable regions of mAb A6 (58), cloned by RT-PCR, with human gamma-1 heavy chain and human kappa light chain constant regions. After transfection into SP2/0 cells and selection of clones using G418 and methotrexate, the antibody was purified by affinity chromatography over goat anti-human IgG followed by size exclusion chromatography. Endotoxin was removed using ACTICLEAN ETOX (Sterogene, 2705-01). Final endotoxin levels were below 30 pg/mg protein.

### Example 2

### Differentiation of DC

PBMCs from healthy donors were isolated by centrifugation over Ficoll-Hypaque gradients (Nycomed Amersham). CD14⁺ monocytes were isolated as the adherent fraction following incubation for 1 hour in RPMI 1640 (Biowhittaker) supplemented with 10% FCS (Biowhittaker), 100 U/ml penicillin/streptomycin (Bristol-Myers Squibb), and 50 μM 2 mercaptoethanol (BioRad) (DC medium) at 37° C. Following extensive washing, adherent monocytes were differentiated into DC by culture in 10 ng/ml rhIL-4 (R&D Systems) and 100 ng/ml rhGM-CSF (Immunotools) in DC medium. After 5 days, DC were either left unstimulated or transferred to wells containing irradiated (10,000 RADS) 3T3 fibroblasts expressing human CD40L to induce maturation. During DC maturation cells were cultured in presence or absence of anti-CD45RO/RB (chA6) mAb (10 μg/ml). After 2 days, immature, mature and mature/chA6 DC were collected, irradiated (6000 RADS) and used to stimulate T-cells, and frozen and thawed before each round of stimulation. The purity and maturation state of DC was routinely checked by flow cytometric analysis to determine expression of CD1a, CD14, CD83 and HLA-DR. Typically the cultures contained >90% CD1a⁺CD14⁻ cells. In some experiments immature, mature and chA6-modulated (mature/chA6) DC were also tested for levels of expression of costimulatory molecules CD40, CD80 and CD86, ICOS-ligand, ILT-4 (kind gifts from Gregorio Aversa), ILT-3 (Immunotech), PDL-1, PDL-2 (eBioscience), ICAM-1, LFA-1, CD45RO and CD45RB (BD bioscience), and SLAM (kind gifts from Gregorio Aversa) expression.

### Example 3

### Purification of T-Cells

CD4⁺ T-cells were purified from PBMCs by negative selection using the CD4⁺ T-cell Isolation kit (Miltenyi Biotech), according to the manufacture's instructions. A portion of the resulting CD4⁺ T-cells was cryopreserved for later use, and the remainders were depleted of CD45RO⁺ cells using anti-CD45RO-coupled magnetic beads and LD negative selection columns (Miltenyi Biotech). The resulting cells were routinely greater than 90% CD4⁺CD45RO⁻CD45RA⁺.

### Example 4

### T-Cell Differentiation

1×10⁵ DC were cultured with 1×10⁶ allogeneic CD4⁺CD45RO⁻T-cells in 2 ml of X-vivo 15 medium (Biowhittaker), supplemented with 5% pooled AB human serum (Biowhittaker), and 100 U/ml penicillin/streptomycin (Bristol-Myers Squibb). After 6 or 7 days, rhIL-2 (20 U/ml) (Chiron) was added, and cells were expanded for an additional 7-8 days. Fourteen days after initiation of the culture, T-cells were collected, washed, and restimulated with immature, mature or mature/chA6 DC from the same allogeneic donor used in the primary culture. After 3 days, rhIL-2 was added. After the second stimulation, T-cells were collected, washed, and tested for their proliferative and suppressive capacity. In some experiments, neutralizing anti-PDL2 (MIH18, 10 μg/ml, eBioscience) mAbs were added at the initiation of each round of stimulation and each time the cells were split. T-cells stimulated repeatedly with immature DC are referred to as T(imm), those stimulated repeatedly with mature DC as T(mat) and those stimulated repeatedly with mature/chA6 DC as T(chA6 mat).

### Example 5

### Proliferation and Suppression of T-Cells

To test for the capacity of T(imm), T(mat) or T(chA6 mat) cells to suppress proliferation and/or cytokine production, autologous CD4⁺ T-cells were thawed and stimulated with either allogeneic mature DC (10:1, T:DC) or monocytes (CD3-depleted PBMCs, irradiated 6000 RADS) (1:1, T:monocytes). Naive CD4⁺ T-cells were stimulated alone, or in the presence of T(imm), T(mat) of T(chA6 mat) cells (1:1 ratio) in a final volume of 200 μl of complete medium in 96 well round-bottom plates. In some cultures anti-IL-10R (30 μg/ml, 3F9) and/or anti-TGF-β (50 μg/ml, 1D11, R&D systems) mAbs were added. After the indicated time, wells were either pulsed for 16 hours with 1 μCi/well ³H-thymidine or supernatants were collected for analysis of IFN-γ production.

### Example 6

### ELISAs

T(imm), T(mat) or T(chA6 mat) were stimulated with mature allogeneic DC at a ratio of 10:1 (T:DC). Supernatants were collected after 24 hours for IL-2 and IL-4, 48 hours for IL-10 and IFN-γ, and 72 hours for TGF-β. To assess the amount of cytokines produced by immature, mature and mature/chA6 DC, DC were cultured alone. Supernatants were harvested after 48 hours. Levels of IL-2, IL-4, IL-10, IL-12, IL-6, TNF-α and IFN-γ were determined by capture ELISA according to the manufacturer's instructions (BD Biosciences). Levels of TGF-β in acidified supernatants were determined by capture ELISA according to the manufacture's instructions (R&D systems). The limits of detection were as follows: IL-2: 20 pg/ml; IL-4: 20 pg/ml; IL-10: 20 pg/ml; IL-12: 30 pg/ml, IL-6: 30 pg/ml, TNF-α: 20 pg/ml IFN-γ: 60 pg/ml; TGF-β: 60 pg/ml.

### Example 7

### Phenotype of chA6 mAb Modulated Mature DC

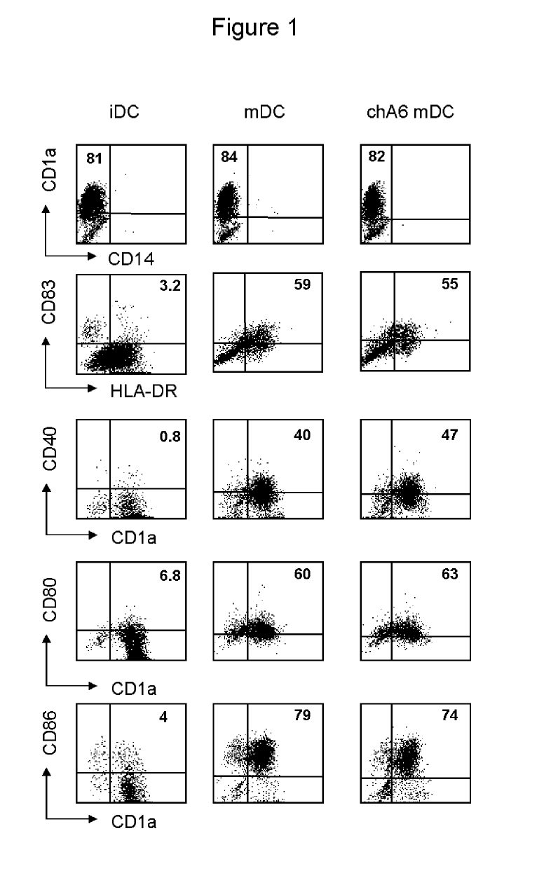

Mature DC generated in the presence of chA6 mAb comprise a mix population of cells consisting in typically mature DC and cells similar to immature DC. To determine whether chA6 treatment modulated the differentiation and maturation status of mature DC, a phenotypic analysis of cells was performed. DC were differentiated from CD14⁺ monocytes in the presence of IL-4 and GM-CSF for 5 days, and then either left unstimulated or activated by co-culture with murine fibroblasts expressing CD40L for 48 hours in the presence or absence of soluble chA6 mAb. As expected, cultures of both immature, mature and mature/chA6 DC were routinely >90% CD1a⁺CD14⁻ (FIG. 1).

Immature DC were CD83 negative and HLA-DR^{low}. Addition of chA6 mAb during the DC activation did not modify the expression of CD83 and HLA-DR, which were up-regulated on mature DC (FIG. 1). Mature/chA6 and mature DC expressed comparable levels of costimulatory molecules CD40, CD80 and CD86.

### Example 8

### chA6 mAb Modified the Expression of PDL-2 and CD45RB on Mature DC

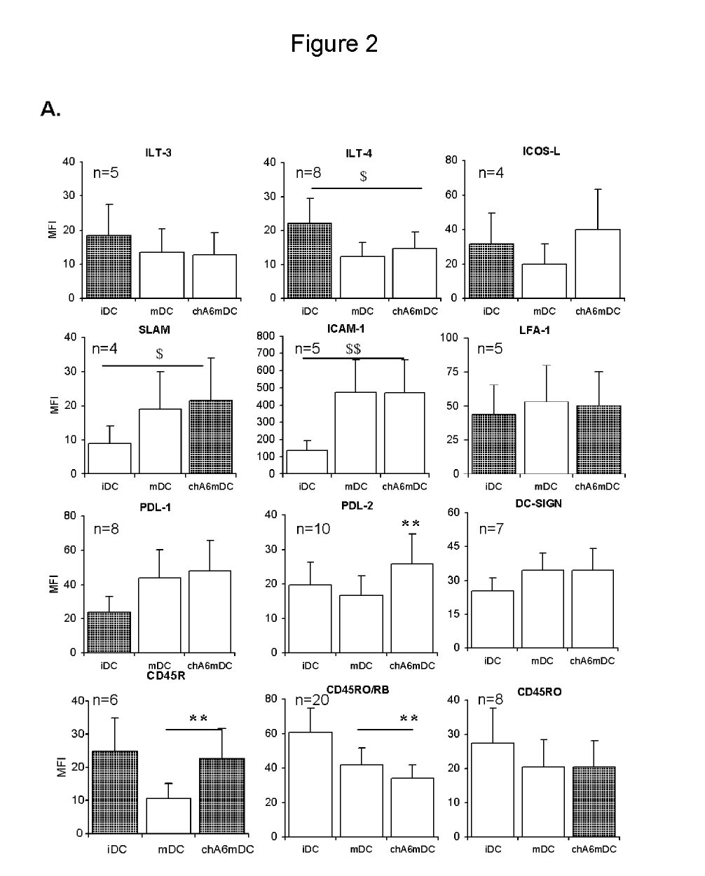

We next determined whether molecules previously associated with tolerogenic DC were expressed by mature/chA6 DC. The expression of ILT3 and ILT4 were similar on mature/chA6 DC and mature DC, and as expected, they were lower compared to immature DC, (FIG. 2A). The MFI of ITL3 was 12.8±6.4 on chA6 mature DC versus 13.6±6.4 on mature DC (n=5, p=ns), and versus 18.4±9.2 on immature DC (n=5, p=ns). The MFI for ILT4 was 14.7±4.9 on mature/chA6 DC versus 12.4±4.1 on mature DC (n=8, p=ns), and versus 22.1±7.4 on immature DC (n=5, p=0.05). The expression of ICOS-L was slightly increased on mature/chA6 DC compared to mature and immature DC: MFI of ICOS-L was 40.1±23.2 on mature/chA6 DC versus 20±11.5 on mature DC (n=4, ns), and versus 31.4±18.1 on immature DC (n=4, p=ns). Mature/chA6 DC and mature DC expressed similar levels of SLAM, with an MFI of 21.5±3.9 on mature/chA6

DC versus 18.9±10.9 on mature DC (n=4, ns), which was significantly higher compared to immature DC (8.9±5.1, n=4, p=0.05). No differences in the expression of adhesion molecules ICAM-1 and LFA-1 were observed between mature/chA6 DC and mature DC: the MFI of ICAM-1 was 471.5±192.5 on mature/chA6 DC versus 472.1±192.7 on mature DC (n=5, p=ns), significantly higher compared to immature DC (136.8±55.9, n=5, p=0.02). The MFI of LFA-1 was 50.3±25.2 on mature/chA6 DC versus 53.3±26.6 on mature DC (n=5, p=ns) and was slightly increased compared to immature DC (43.7±21.9, n=5, p=n.s.). The expression of PDL-1 was comparable in mature/chA6 and mature DC, and was significantly higher compared to immature DC, as previously reported (59). The MFI of PDL-1 on mature/chA6 DC was 43.8±16.6 compared to 47.9±18.1 on mature DC (n=8, p ns), and to 25.9±9 on immature DC (n=8, p≦0.001). The expression of DC-SIGN was comparable on mature/chA6 and mature DC, but slightly higher compared to immature DC. The MFI of DC-SIGN was 34.4±9.6, 34.3±7.8, and 25.2±5.8, on mature/chA6, mature, and immature DC, respectively. In contrast, the expression of PDL-2 was significantly higher on mature/chA6 DC (FIG. 2). The MFI of PDL-2 on mature/chA6 DC was 25.8±8.6 versus 16.8±5.6 on mature DC (n=10, p=0.009), and versus 19.7±6.6 on immature DC (n=10, p=ns). We also demonstrated that the expression of CD45RB was higher on DC matured in the presence of chA6 mAb. The MFI of CD45RB was 22.6±9.2 on mature/chA6 DC versus 10.7±4.4 on mature DC (n=6, p=0.05), and versus 24.8±10.1 (n=6, p=0.04) on immature DC. In contrast, the expression of CD45RO/RB isoform was significantly lower on mature/chA6 DC compared to mature and immature DC. The MFI of CD45RO/RB was 34.1±7.8 on mature/chA6 DC versus 41.9±9.6 on mature DC (n=20, p=0.01), and 60.6±13.9 on immature DC (n=20, p=0.02). The down-regulation of CD45RO/RB isoform was not due to the presence of chA6 mAb, since staining of mature/chA6 DC with a secondary antibody was similar to staining with isotype control (data not shown). The expression of CD45RO isoform was comparable among the three subset of DC. The MFI of CD45RO was 27.3±10.3, 20.5±7.8, and 20.4±7.7, on immature, mature, and mature/chA6 DC, respectively.

### Example 9

### ChA6 mAb Treatment does not Modify Cytokine Production Profile of Mature DC

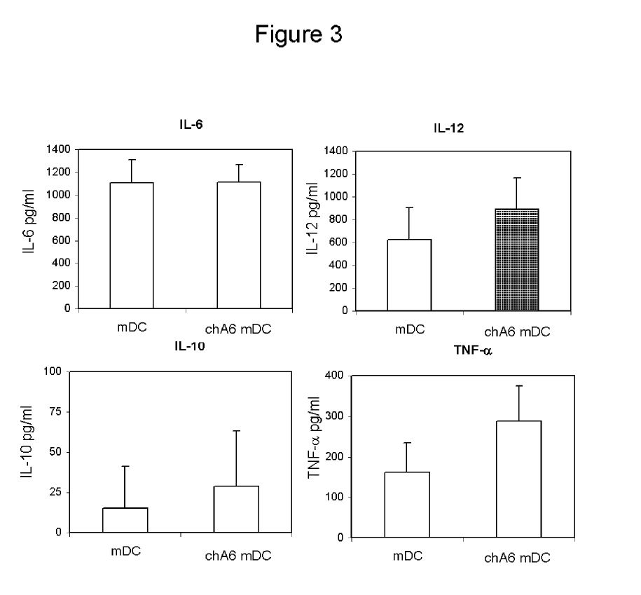

We next determined the cytokine secretion profile of DC. Immature, mature, and mature/chA6 DC were washed after 7 day of culture and re-plated for two additional days. Mature/chA6 DC secreted similar amounts of IL-6, IL-12, IL-10, and TNF-α compared to mature DC. (FIG. 3). Together these results indicate that addition of chA6 during maturation of DC does not modify the cytokine production of the resulting mature DC. These results do not exclude the possibility that the expression and secretion of other cytokines that we did not analyze can be modulated by chA6 mAbs.

### Example 10

### chA6 mAb Induce Tolerogenic DC

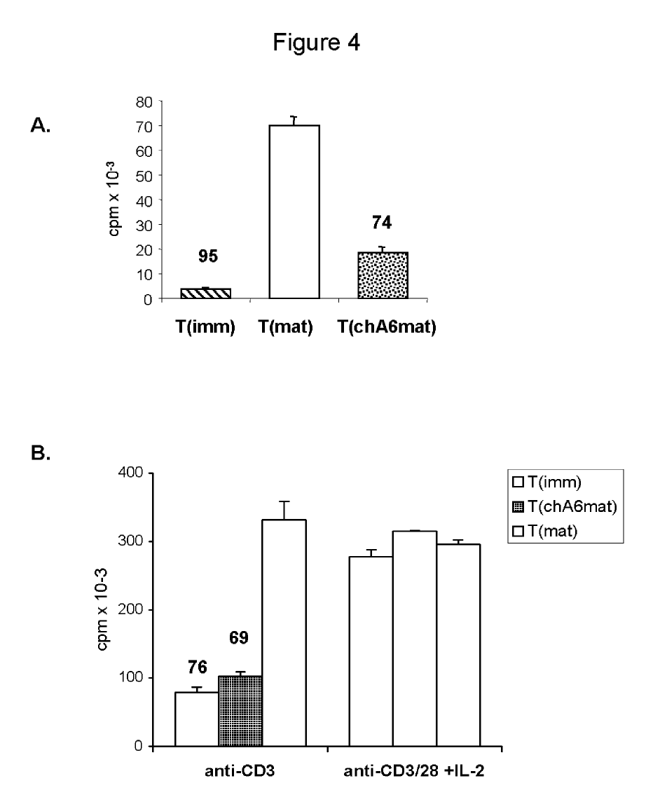

We then investigated whether mature/chA6 DC were as efficient as immature DC in generating Tr cells in vitro. CD4+CD45RO- T-cells were repetitively stimulated (3 rounds of stimulation) with allogeneic mature/chA6 DC at a 10:1 ratio, using our standardized protocol (38), and subsequently tested for their ability to proliferate in response to mature DC. Surprisingly, after 3 rounds of stimulation, T-cells primed with allogeneic mature/chA6 DC were hypo-responsive to re-activation with fully mature DC (FIG. 4A). An average reduction of 57±23% (n=17, p=0.009) in Ag-induced proliferation was observed in comparison to T-cells stimulated with mature DC. As expected, T-cells primed with allogeneic immature DC were hypo-responsive to re-activation with allogeneic mature DC with an average reduction of proliferation of 75±17% (n=23, p=0.0009) in comparison to T-cells repetitively primed with mature DC. Similar results were obtained in response to polyclonal activation (FIG. 4B), with an average reduction in proliferation of 62.6±16.5% (n=3) after three rounds of activation with mature/chA6 DC and of 78.7±20% with immature DC (n=3). This hypo-responsiveness could be rescued by addition of anti-CD28 mAb and exogenous IL-2 (FIG. 4B).

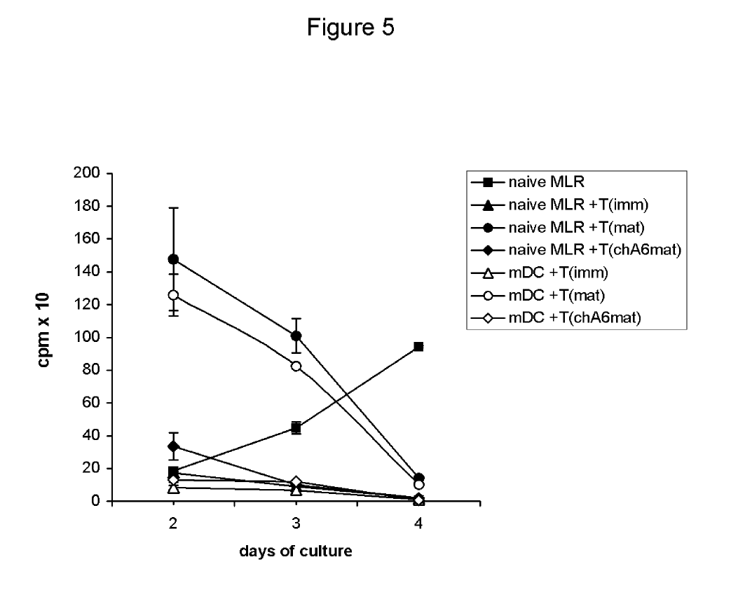

The finding that repetitive in vitro stimulation of peripheral blood CD4+CD45RO- T-cells with mature/chA6 DC resulted in profoundly hyporesponsive T-cells suggested that these cells might also have acquired suppressive capacity. We therefore tested the ability of T-cells generated with mature/chA6 DC to suppress the responses of nave autologous CD4+ T-cells upon challenge with allogeneic mature DC. Naïve CD4+ T-cells were stimulated with mature DC alone, or in the presence of T(chA6 mat) or T(mat) cells (1:1 ratio), and proliferation was assessed 2, 3 or 4 days after initiation of the culture. As control, naïve CD4+ T-cells primed with mature DC were co-cultured with T(imm) cells. Naïve CD4+ T-cells stimulated with mature DC displayed the kinetics of a primary response, with proliferation peaking after 4 days of culture (FIG. 5). As expected, T(mat) cells generated with mature DC, displayed kinetics of a secondary response when re-challenged with DC from the same donor, with proliferation peaking at day 2. T(chA6 mat) cells remained hyporesponsive throughout the time course. Addition of T(mat) cells to the primary MLR resulted in increased proliferation at day 2. Importantly, addition of both T(chA6 mat) or T(imm) cells suppressed proliferation of naïve CD4+ T-cells in response to mature DC. An average reduction of 76±23% and of 87±10% (n=13) in proliferation of naïve CD4+ T-cells was observed when assessed 4 days after culture with T(chA6 mat) cells and T(imm) cells, respectively. Together, these results indicate that activation of DC in the presence of chA6 mAb results in the generation of tolerogenic DC, which induce Tr cells in vitro.

### Example 11

### T-Cells Generated by chA6-Modulated DC are Phenotypically and Functionally Equivalent to Tr1 Cells

We next examined whether Tr cells induced by repetitively stimulation with mature/chA6 DC were similar to IL-10-producing Tr 1 cells. We first determined the cytokine production profile of T(chA6mat) cells following activation with mature DC, and we compared their cytokine production profile to either T(imm) or T(mat) cells. As shown in Table 1, T(mat) cells produced all cytokines tested. In contrast, T(chA6 mat) cells produced IL-10, IFN-γ and TGF-β, and failed to produce significant levels of IL-2 or IL-4. Similarly to T(imm) cells, T(chA6mat) cells produced slightly lower amounts of IL-10 in comparison to T(mat) cells, and levels of TGF-β were not significantly different. T(chA6 mat) cells produce IFN-γ, but at least 10 fold less compared to that secreted by T(mat) cells. Therefore, T(chA6mat) cells display a cytokine production profile similar to that of Tr1 cells.

TABLE 1
Cytokine production profile of Timm, Tmat, and TchA6maT-cells.

| | T₍ᵢₘₘ₎ | T₍ₘₐₜ₎ | T_{(chA6 mat)} | P |
|---|---|---|---|---|
| IL-2, pg/ml | <20 | 1500 ± 500 | <20 | 0.02 |
| IL-4, pg/ml | <20 | 130 ± 60 | <20 | ns |
| IL-10, pg/ml | 430 ± 65 | 800 ± 170 | 600 ± 120 | 0.05 |
| IFN-γ, ng/ml | 1.3 ± 0.5 | 6.5 ± 2.7 | 0.8 ± 0.2 | 0.05 |
| TGF-β, pg/ml | 410 ± 200 | 400 ± 200 | 350 ± 160 | ns |

At the end of 3 rounds of stimulation with immature, mature and chA6/mature DC, T-cell lines were activated with mDC and supernatants were collected after 24 h (for IL-2), 48 h (for IL-10, IFN-□, and TGF-□) of culture. Levels of the indicated cytokines were determined by ELISA. The average ± SEM amounts detected in eight independent experiments are presented.

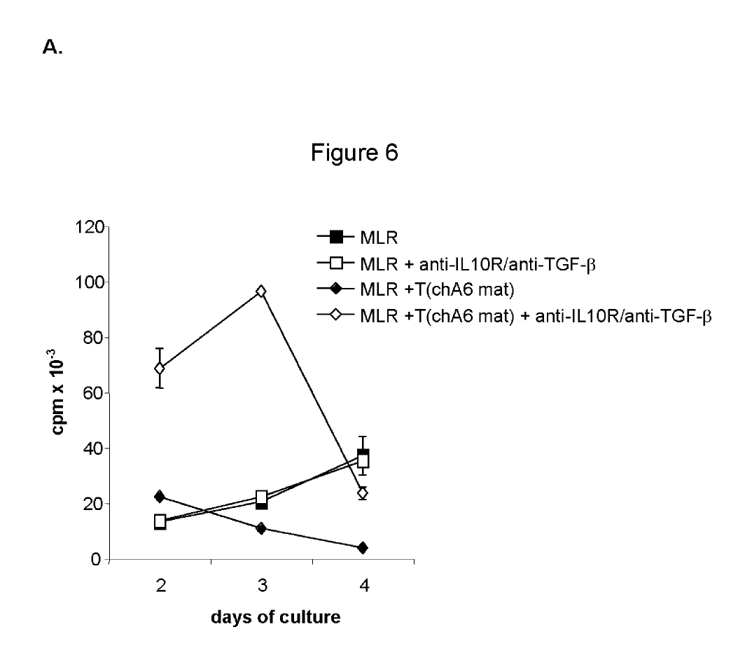

We next investigated whether suppression of proliferation by T(chA6 mat) cells was mediated via production of IL-10 and/or TGF-β. We performed suppression experiments using allogeneic monocytes to induce proliferation of nave T-cells. Under these conditions, addition of neutralizing anti-IL-10R and anti-TGF-β mAbs completely reversed suppression of proliferation mediated by T(chA6mat) cells (FIG. 6). Together, these data indicate that Tr cells generated by repetitive stimulation with mature/chA6 DC are phenotypically and functionally equivalent to Tr1 cells.

### Example 12

### Differentiation of Tr1 Cells by chA6 Mature DC Requires PDL-2/PD-1 Interaction

We showed that among the tolerogenic markers tested, PDL-2 was significantly up-regulated on mature DC treated with chA6 mAb. PDL-2 is known to be an inhibitory receptor, selectively expressed by DC (59). We therefore investigated whether PDL-2/PD-1 interaction was required for the generation of Tr 1 cells induced by mature/chA6 DC. CD4+CD45RO- T-cells were stimulated repetitively with mature/chA6 DC in the absence or presence of neutralizing anti-PDL-2 or control IgG mAbs. As shown in FIG. 8A, differentiation of T-cells in the presence of neutralizing anti-PDL-2 mAbs completely reversed the hyporesponsive state induced by mature/chA6 DC. Moreover, PDL-2 blockade also prevented the induction of Tr cells with suppressive activity (FIG. 8B).

### REFERENCES

1. Bluestone, J. A., J. B. Matthews, and A. M. Krensky. 2000. The immune tolerance network: the “Holy Grail” comes to the clinic. J Am Soc Nephrol 11:2141-2146.
2. Lenschow, D. J., Y. Zeng, J. R. Thistlethwaite, A. Montag, W. Brady, M. G. Gibson, P. S. Linsley, and J. A. Bluestone. 1992. Long-term survival of xenogeneic pancreatic islet grafts induced by CTLA41g. Science 257:789-792.
3. Bushell, A., P. J. Morris, and K. J. Wood. 1995. Transplantation tolerance induced by antigen pretreatment and depleting anti-CD4 antibody depends on CD4+ T-cell regulation during the induction phase of the response. Eur J Immunol 25:2643-2649.
4. Larsen, C. P., E. T. Elwood, D. Z. Alexander, S. C. Ritchie, R. Hendrix, C. Tucker-Burden, H. R. Cho, A. Aruffo, D. Hollenbaugh, P. S. Linsley, K. J. Winn, and T. C. Pearson. 1996. Long-term acceptance of skin and cardiac allografts after blocking CD40 and CD28 pathways. Nature 381:434-438.
5. Larsen, C. P., S. J. Knechtle, A. Adams, T. Pearson, and A. D. Kirk. 2006. A new look at blockade of T-cell costimulation: a therapeutic strategy for long-term maintenance immunosuppression. Am J Transplant 6:876-883.
6. Li, Y., X. C. Li, X. X. Zheng, A. D. Wells, L. A. Turka, and T. B. Strom. 1999. Blocking both signal 1 and signal 2 of T-cell activation prevents apoptosis of alloreactive T-cells and induction of peripheral allograft tolerance. Nat Med 5:1298-1302.
7. Woodle, E. S., D. Xu, R. A. Zivin, J. Auger, J. Charette, R. O'Laughlin, D. Peace, L. K. Jollife, T. Haverty, J. A. Bluestone, and J. R. Thistlethwaite, Jr. 1999. Phase I trial of a humanized, Fc receptor nonbinding OKT3 antibody, huOKT3gamma1 (Ala-Ala) in the treatment of acute renal allograft rejection. Transplantation 68:608-616.
8. Friend, P. J., G. Hale, L. Chatenoud, P. Rebello, J. Bradley, S. Thiru, J. M. Phillips, and H. Waldmann. 1999. Phase I study of an engineered aglycosylated humanized CD3 antibody in renal transplant rejection. Transplantation 68:1632-1637.
9. Herold, K. C., S. E. Gitelman, U. Masharani, W. Hagopian, B. Bisikirska, D. Donaldson, K. Rother, B. Diamond, D. M. Harlan, and J. A. Bluestone. 2005. A single course of anti-CD3 monoclonal antibody hOKT3gamma1 (Ala-Ala) results in improvement in C-peptide responses and clinical parameters for at least 2 years after onset of type 1 diabetes. Diabetes 54:1763-1769.
10. Keymeulen, B., E. Vandemeulebroucke, A. G. Ziegler, C. Mathieu, L. Kaufman, G. Hale, F. Gorus, M. Goldman, M. Walter, S. Candon, L. Schandene, L. Crenier, C. De Block, J. M. Seigneurin, P. De Pauw, D. Pierard, I. Weets, P. Rebello, P. Bird, E. Berrie, M. Frewin, H. Waldmann, J. F. Bach, D. Pipeleers, and L. Chatenoud. 2005. Insulin needs after CD3-antibody therapy in new-onset type 1 diabetes. N Engl J Med 352:2598-2608
11. Utset, T. O., J. A. Auger, D. Peace, R. A. Zivin, D. Xu, L. Jolliffe, M. L. Alegre, J. A. Bluestone, and M. R. Clark. 2002. Modified anti-CD3 therapy in psoriatic arthritis: a phase I/II clinical trial. J Rheumatol 29:1907-1913.
12. Cortesini, R., and N. Suciu-Foca. 2004. The concept of “partial” clinical tolerance. Transpl Immunol 13:101-104.
13. Watanabe, T., J. I. Masuyama, Y. Sohma, H. Inazawa, K. Horie, K. Kojima, Y. Uemura, Y. Aoki, S. Kaga, S. Minota, T. Tanaka, Y. Yamaguchi, T. Kobayashi, and I. Serizawa. 2006. CD52 is a novel costimulatory molecule for induction of CD4(+) regulatory T-cells. Clin Immunol.
14. Kirk, A. D., L. C. Burkly, D. S. Batty, R. E. Baumgartner, J. D. Berning, K. Buchanan, J. H. Fechner, Jr., R. L. Germond, R. L. Kampen, N. B. Patterson, S. J. Swanson, D. K. Tadaki, C. N. TenHoor, L. White, S. J. Knechtle, and D. M. Harlan. 1999. Treatment with humanized monoclonal antibody against CD154 prevents acute renal allograft rejection in nonhuman primates. Nat Med 5:686-693.
15. Kenyon, N. S., M. Chatzipetrou, M. Masetti, A. Ranuncoli, M. Oliveira, J. L. Wagner, A. D. Kirk, D. M. Harlan, L. C. Burkly, and C. Ricordi. 1999. Long-term survival and function of intrahepatic islet allografts in rhesus monkeys treated with humanized anti-CD154. Proc Natl Acad Sci USA 96:8132-8137.
16. Kenyon, N. S., L. A. Fernandez, R. Lehmann, M. Masetti, A. Ranuncoli, M. Chatzipetrou, G. Iaria, D. Han, J. L. Wagner, P. Ruiz, M. Berho, L. Inverardi, R. Alejandro, D. H. Mintz, A. D. Kirk, D. M. Harlan, L. C. Burkly, and C. Ricordi. 1999. Long-term survival and function of intrahepatic islet allografts in baboons treated with humanized anti-CD154. Diabetes 48:1473-1481.
17. Kawai, T., D. Andrews, R. B. Colvin, D. H. Sachs, and A. B. Cosimi. 2000. Thromboembolic complications after treatment with monoclonal antibody against CD40 ligand. Nat Med 6:114.
18. Xu, H., S. P. Montgomery, E. H. Preston, D. K. Tadaki, D. A. Hale, D. M. Harlan, and A. D. Kirk. 2003. Studies investigating pretransplant donor-specific blood transfusion, rapamycin, and the CD154-specific antibody IDEC-131 in a nonhuman primate model of skin allotransplantation. J Immunol 170:2776-2782.
19. Preston, E. H., H. Xu, K. K. Dhanireddy, J. P. Pearl, F. V. Leopardi, M. F. Starost, D. A. Hale, and A. D. Kirk. 2005. IDEC-131 (anti-CD154), sirolimus and donor-specific transfusion facilitate operational tolerance in non-human primates. Am J Transplant 5:1032-1041.
20. Moore, K. W., R. de Waal Malefyt, R. L. Coffman, and A. O'Garra. 2001. Interleukin-10 and the interleukin-10 receptor. Annu Rev Immunol 19:683-765.
21. Fiorentino, D. F., A. Zlotnik, P. Vieira, T. R. Mosmann, M. Howard, K. W. Moore, and A. O'Garra. 1991. IL-10 acts on the antigen-presenting cell to inhibit cytokine production by Th1 cells. J Immunol 146:3444-3451.
22. Willems, F., A. Marchant, J. P. Delville, C. Gerard, A. Delvaux, T. Velu, M. de Boer, and M. Goldman. 1994. Interleukin-10 inhibits B7 and intercellular adhesion molecule-1 expression on human monocytes. Eur J Immunol 24:1007-1009.
23. Fiorentino, D. F., A. Zlotnik, T. R. Mosmann, M. Howard, and A. O'Garra. 1991. IL-10 inhibits cytokine production by activated macrophages. J Immunol 147:3815-3822.
24. de Waal Malefyt, R., J. Haanen, H. Spits, M. G. Roncarolo, A. to Velde, C. Figdor, K. Johnson, R. Kastelein, H. Yssel, and J. E. de Vries. 1991. Interleukin 10 (IL-10) and viral IL-10 strongly reduce antigen-specific human T-cell proliferation by diminishing the antigen-presenting capacity of monocytes via downregulation of class II major histocompatibility complex expression. J Exp Med 174:915-924.
25. Allavena, P., L. Piemonti, D. Longoni, S. Bernasconi, A. Stoppacciaro, L. Ruco, and A. Mantovani. 1998. IL-10 prevents the differentiation of monocytes to dendritic cells but promotes their maturation to macrophages. Eur J Immunol 28:359-369.
26. Groux, H., A. O'Garra, M. Bigler, M. Rouleau, S. Antonenko, J. E. de Vries, and M. G. Roncarolo. 1997. A CD4+ T-cell subset inhibits antigen-specific T-cell responses and prevents colitis. Nature 389:737-742.
27. Zeller, J. C., A. Panoskaltsis-Mortari, W. J. Murphy, F. W. Ruscetti, S. Narula, M. G. Roncarolo, and B. R. Blazar. 1999. Induction of CD4+ T-cell alloantigen-specific hyporesponsiveness by IL-10 and TGF-beta. J Immunol 163:3684-3691.
28. Groux, H., M. Bigler, J. E. de Vries, and M. G. Roncarolo. 1996. Interleukin-10 induces a long-term antigen-specific anergic state in human CD4+ T-cells. J Exp Med 184:19-29.
29. Bacchetta, R., M. Bigler, J. L. Touraine, R. Parkman, P. A. Tovo, J. Abrams, R. de Waal Malefyt, J. E. de Vries, and M. G. Roncarolo. 1994. High levels of interleukin 10 production in vivo are associated with tolerance in SCID patients transplanted with HLA mismatched hematopoietic stem cells. J Exp Med 179:493-502.
30. Boussiotis, V. A., Z. M. Chen, J. C. Zeller, W. J. Murphy, A. Berezovskaya, S. Narula, M. G. Roncarolo, and B. R. Blazar. 2001. Altered T-cell receptor+CD28-mediated signaling and blocked cell cycle progression in interleukin 10 and transforming growth factor-beta-treated alloreactive T-cells that do not induce graft-versus-host disease. Blood 97:565-571.
31. Banchereau, J., F. Briere, C. Caux, J. Davoust, S. Lebecque, Y. J. Liu, B. Pulendran, and K. Palucka. 2000. Immunobiology of dendritic cells. Annu Rev Immunol 18:767-811.
32. Roncarolo, M. G., M. K. Levings, and C. Traversari. 2001. Differentiation of T regulatory cells by immature dendritic cells. J Exp Med 193:F5-9.
33. Hawiger, D., K. Inaba, Y. Dorsett, M. Guo, K. Mahnke, M. Rivera, J. V. Ravetch, R. M. Steinman, and M. C. Nussenzweig. 2001. Dendritic cells induce peripheral T-cell unresponsiveness under steady state conditions in vivo. J Exp Med 194:769-779.
34. Bonifaz, L., D. Bonnyay, K. Mahnke, M. Rivera, M. C. Nussenzweig, and R. M. Steinman. 2002. Efficient targeting of protein antigen to the dendritic cell receptor DEC-205 in the steady state leads to antigen presentation on major histocompatibility complex class I products and peripheral CD8+ T-cell tolerance. J Exp Med 196:1627-1638.
35. Dhodapkar, M. V., R. M. Steinman, J. Krasovsky, C. Munz, and N. Bhardwaj. 2001. Antigen-specific inhibition of effector T-cell function in humans after injection of immature dendritic cells. J Exp Med 193:233-238.
36. Mahnke, K., E. Schmitt, L. Bonifaz, A. H. Enk, and H. Jonuleit. 2002. Immature, but not inactive: the tolerogenic function of immature dendritic cells. Immunol Cell Biol 80:477-483.
37. Jonuleit, H., E. Schmitt, G. Schuler, J. Knop, and A. H. Enk. 2000. Induction of interleukin 10-producing, nonproliferating CD4(+) T-cells with regulatory properties by repetitive stimulation with allogeneic immature human dendritic cells. J Exp Med 192:1213-1222.
38. Levings, M. K., S. Gregori, E. Tresoldi, S. Cazzaniga, C. Bonini, and M. G. Roncarolo. 2005. Differentiation of Tr1 cells by immature dendritic cells requires IL-10 but not CD25+CD4+Tr cells. Blood 105:1162-1169.
39. Sallusto, F., and A. Lanzavecchia. 1999. Mobilizing dendritic cells for tolerance, priming, and chronic inflammation. J Exp Med 189:611-614.
40. Woltman, A. M., and C. van Kooten. 2003. Functional modulation of dendritic cells to suppress adaptive immune responses. J Leukoc Biol 73:428-441.
41. Steinbrink, K., E. Graulich, S. Kubsch, J. Knop, and A. H. Enk. 2002. CD4(+) and CD8(+) anergic T-cells induced by interleukin-10-treated human dendritic cells display antigen-specific suppressor activity. Blood 99:2468-2476.
42. Steinbrink, K., M. Wolff, H. Jonuleit, J. Knop, and A. H. Enk. 1997. Induction of tolerance by IL-10-treated dendritic cells. J Immunol 159:4772-4780.
43. Sato, K., N. Yamashita, M. Baba, and T. Matsuyama. 2003. Modified myeloid dendritic cells act as regulatory dendritic cells to induce anergic and regulatory T-cells. Blood 101:3581-3589.
44. Carbonneil, C., H. Saidi, V. Donkova-Petrini, and L. Weiss. 2004. Dendritic cells generated in the presence of interferon-alpha stimulate allogeneic CD4+ T-cell proliferation: modulation by autocrine IL-10, enhanced T-cell apoptosis and T regulatory type 1 cells. Int Immunol 16:1037-1052.
45. Ito, T., R. Amakawa, M. Inaba, S. Ikehara, K. Inaba, and S. Fukuhara. 2001. Differential regulation of human blood dendritic cell subsets by IFNs. J Immunol 166:2961-2969.
46. Menges, M., S. Rossner, C. Voigtlander, H. Schindler, N. A. Kukutsch, C. Bogdan, K. Erb, G. Schuler, and M. B. Lutz. 2002. Repetitive injections of dendritic cells matured with tumor necrosis factor alpha induce antigen-specific protection of mice from autoimmunity. J Exp Med 195:15-21.
47. Bottomly, K., M. Luqman, L. Greenbaum, S. Carding, J. West, T. Pasqualini, and D. B. Murphy. 1989. A monoclonal antibody to murine CD45R distinguishes CD4 T-cell populations that produce different cytokines Eur J Immunol 19:617-623.
48. Lee, W. T., X. M. Yin, and E. S. Vitetta. 1990. Functional and ontogenetic analysis of murine CD45Rhi and CD45Rlo CD4+ T-cells. J Immunol 144:3288-3295.
49. Ashwell, J. D., and U. D'Oro. 1999. CD45 and Src-family kinases: and now for something completely different. Immunol Today 20:412-416.
50. Penninger, J. M., J. Irie-Sasaki, T. Sasaki, and A. J. Oliveira-dos-Santos. 2001. CD45: new jobs for an old acquaintance. Nat Immunol 2:389-396.
51. Lazarovits, A. I., S. Poppema, Z. Zhang, M. Khandaker, C. E. Le Feuvre, S. K. Singhal, B. M. Garcia, N. Ogasa, A. M. Jevnikar, M. H. White, G. Singh, C. R. Stiller, and R. Z. Zhong. 1996. Prevention and reversal of renal allograft rejection by antibody against CD45RB. Nature 380:717-720.
52. Basadonna, G. P., L. Auersvald, C. Q. Khuong, X. X. Zheng, N. Kashio, D. Zekzer, M. Minozzo, H. Qian, L. Visser, A. Diepstra, A. I. Lazarovits, S. Poppema, T. B. Strom, and D. M. Rothstein. 1998. Antibody-mediated targeting of CD45 isoforms: a novel immunotherapeutic strategy. Proc Natl Acad Sci USA 95:3821-3826.
53. Fecteau, S., G. P. Basadonna, A. Freitas, C. Ariyan, M. H. Sayegh, and D. M. Rothstein. 2001. CTLA-4 up-regulation plays a role in tolerance mediated by CD45. Nat Immunol 2:58-63.
54. Ariyan, C., P. Salvalaggio, S. Fecteau, S. Deng, L. Rogozinski, D. Mandelbrot, A. Sharpe, M. H. Sayegh, G. P. Basadonna, and D. M. Rothstein. 2003. Cutting edge: transplantation tolerance through enhanced CTLA-4 expression. J Immunol 171:5673-5677.
55. Salvalaggio, P. R., G. Camirand, C. E. Ariyan, S. Deng, L. Rogozinski, G. P. Basadonna, and D. M. Rothstein. 2006. Antigen exposure during enhanced CTLA-4 expression promotes allograft tolerance in vivo. J Immunol 176:2292-2298.
56. Deng, S., D. J. Moore, X. Huang, M. Mohiuddin, M. K. t. Lee, E. Velidedeoglu, M. M. Lian, M. Chiaccio, S. Sonawane, A. Orlin, J. Wang, H. Chen, A. Caton, R. Zhong, and J. F. Markmann. 2006. Antibody-induced transplantation tolerance that is dependent on thymus-derived regulatory T-cells. J Immunol 176:2799-2807.
57. Gregori, S., P. Mangia, R. Bacchetta, E. Tresoldi, F. Kolbinger, C. Traversari, J. M. Carballido, J. E. de Vries, U. Korthauer, and M. G. Roncarolo. 2005. An anti-CD45RO/RB monoclonal antibody modulates T-cell responses via induction of apoptosis and generation of regulatory T-cells. J Exp Med 201:1293-1305.
58. Aversa, G., J. A. Waugh, and B. M. Hall. 1994. A monoclonal antibody (A6) recognizing a unique epitope restricted to CD45RO and RB isoforms of the leukocyte common antigen family identifies functional T-cell subsets. Cell Immunol 158:314-328.
59. Brown, J. A., D. M. Dorfman, F. R. Ma, E. L. Sullivan, O. Munoz, C. R. Wood, E. A. Greenfield, and G. J. Freeman. 2003. Blockade of programmed death-1 ligands on dendritic cells enhances T-cell activation and cytokine production. J Immunol 170:1257-1266.

1.-34. (canceled)

35. A composition comprising a CD45 RO/RB binding molecule for use in modulating dendritic cell (DC) function.

36. The composition of claim 35, wherein said binding molecule comprises in sequence the hypervariable regions CDR1, CDR2 and CDR3, said CDR1 having the amino acid sequence Asn-Tyr-Ile-Ile-His (NYIIH), said CDR2 having the amino acid sequence Tyr-Phe-Asn-Pro-Tyr-Asn-His-Gly-Thr-Lys-Tyr-Asn-Glu-Lys-Phe-Lys-Gly (YFNPYNHGTKYNEKFKG) and said CDR3 having the amino acid sequence Ser-Gly-Pro-Tyr-Ala-Trp-Phe-Asp-Thr (SGPYAWFDT).

37. The composition of claim 35, wherein the binding molecule comprises:
a) a first domain comprising in sequence the hypervariable regions CDR1, CDR2 and CDR3, said CDR1 having the amino acid sequence Asn-Tyr-Ile-Ile-His (NYIIH), said CDR2 having the amino acid sequence Tyr-Phe-Asn-Pro-Tyr-Asn-His-Gly-Thr-Lys-Tyr-Asn-Glu-Lys-Phe-Lys-Gly (YFNPYNHGTKYNEKFKG) and said CDR3 having the amino acid sequence Ser-Gly-Pro-Tyr-Ala-Trp-Phe-Asp-Thr (SGPYAWFDT); and
b) a second domain comprising in sequence the hypervariable regions CDR1′, CDR2′ and CDR3′, CDR1′ having the amino acid sequence Arg-Ala-Ser-Gln-Asn-Ile-Gly-Thr-Ser-Ile-Gln (RASQNIGTSIQ), CDR2′ having the amino acid sequence Ser-Ser-Ser-Glu-Ser-Ile-Ser (SSSESIS) and CDR3′ having the amino acid sequence Gln-Gln-Ser-Asn-Thr-Trp-Pro-Phe-Thr (QQSNTWPFT).

38. The composition according to claim 35, wherein the binding molecule is a chimeric or humanized molecule.

39. The composition according to claim 35, wherein the binding molecule is a chimeric or humanized monoclonal antibody, e.g. of the IgG1 isotype.

40. The composition according to claim 35, wherein the binding molecule comprises a polypeptide of SEQ ID NO: 1 and/or a polypeptide of SEQ ID NO:2

41. The composition according to claim 35, wherein the binding molecule comprises a polypeptide of SEQ ID NO: 3 and/or a polypeptide of SEQ ID NO:4.

42. The composition according to claim 35, wherein the binding molecule is a humanized antibody comprising a polypeptide of SEQ ID NO: 9 or of SEQ ID NO: 10 and/or a polypeptide of SEQ ID NO: 7 or of SEQ ID NO: 8.

43. The composition according to claim 35, wherein the binding molecule is a humanized antibody comprising a polypeptide of SEQ ID NO: 31 or of SEQ ID NO: 32 and/or a polypeptide of SEQ ID NO: 7 or of SEQ ID NO: 8.

44. The composition according to claim 35, wherein the binding molecule is a humanized antibody comprising:
(a) a polypeptide of SEQ ID NO: 9 and a polypeptide of SEQ ID NO:7;
(b) a polypeptide of SEQ ID NO: 9 and a polypeptide of SEQ ID NO:8;
(c) a polypeptide of SEQ ID NO: 10 and a polypeptide of SEQ ID NO:7;
(d) a polypeptide of SEQ ID NO: 10 and a polypeptide of SEQ ID NO:8;
(e) a polypeptide of SEQ ID NO: 31 and a polypeptide of SEQ ID NO:7;
(f) a polypeptide of SEQ ID NO: 31 and a polypeptide of SEQ ID NO:8;
(g) a polypeptide of SEQ ID NO: 32 and a polypeptide of SEQ ID NO:7; or
(h) a polypeptide of SEQ ID NO: 32 and a polypeptide of SEQ ID NO:8.

45. The composition according to claim 35, wherein the use is performed in vitro.

46. The composition according to claim 35, for use in inducing the dendritic cells to exhibit a tolerogenic phenotype.

47. A method of maturing dendritic cells in vitro, the method comprising the steps of:
(a) obtaining a source of immature dentritic cells; and
(b) inducing maturation of the immature dentritic cells in the presence of the composition according to claim 35.

48. The method according to claim 47, wherein the dendritic cells are derived from a biological sample.

49. The method according to claim 47, wherein the dendritic cells are obtained by inducing in vitro differentiation of a source of monocytes, e.g. from a biological sample.

50. The method according to claim 47, further comprising the step of exposing the dendritic cells in vitro to a population of T-cells so as to induce a tolerogenic phenotype in said T-cells to produce a population of tolerogenic T-cells.

51. The method according to claim 50, wherein the T-cells are allogeneic with respect to the dendritic cells.

52. A pharmaceutical composition comprising a population of tolerogenic dendritic cells obtained from a method according to claim 47.

53. The pharmaceutical composition according to claim 52, additionally comprising a CD45 RO/RB binding molecule for use in modulating dendritic cell (DC) function.

54. The pharmaceutical composition of claim 52, for use in the treatment and/or prophylaxis of disease associated with autoimmune disease, transplant rejection, psoriasis, inflammatory bowel disease and allergies.

55. A pharmaceutical composition comprising a population of tolerogenic T-cells obtained from a method according to claim 50.

56. The pharmaceutical composition of claim 21, further comprising a CD45 RO/RB binding molecule for use in modulating dendritic cell (DC) function.

57. The pharmaceutical composition of claim 21, further comprising a population of tolerogenic dendrite cells.

58. The pharmaceutical composition of claim 21, further comprising a population of tolerogenic dendrite cells and a CD45 RO/RB binding molecule for use in modulating dendritic cell (DC) function.

59. The pharmaceutical composition of claim 21, for use in the treatment and/or prophylaxis of disease associated with autoimmune disease, transplant rejection, psoriasis, inflammatory bowel disease and allergies.


SEQUENCE LISTING (41 sequences)

SEQ ID NO: 1 - protein, 107 aa, Artificial sequence
  Source 1..107: /note= “Description of artificial sequence Part of the amino acid sequence of chimeric light chain”
DILLTQSPAILSVSPGERVSFSCRASQNIGTSIQWYQQRTNGSPRLLIRSSSESISGIPSRFSGSGSGTDFTLSINSVESEDIADYYCQQSNTWPFTFGSGTKLEIK

SEQ ID NO: 2 - protein, 118 aa, Artificial sequence
  Source 1..118: /note= “Description of artificial sequence Part of the amino acid sequence of chimeric heavy chain”
EVQLQQSGPELVKPGASVKMSCKASGYTFTNYIIHWVKQEPGQGLEWIGYFNPYNHGTKYNEKFKGRATLTADKSSNTAYMDLSSLTSEDSAIYYCARSGPYAWFDTWGQGTTVTVSS

SEQ ID NO: 3 - protein, 214 aa, Artificial sequence
  Source 1..214: /note= “Description of artificial sequence Amino acid sequence of chimeric light chain”
DILLTQSPAILSVSPGERVSFSCRASQNIGTSIQWYQQRTNGSPRLLIRSSSESISGIPSRFSGSGSGTDFTLSINSVESEDIADYYCQQSNTWPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 448 aa, Artificial sequence
  Source 1..448: /note= “Description of artificial sequence Amino acid sequence of chimeric heavy chain”
EVQLQQSGPELVKPGASVKMSCKASGYTFTNYIIHWVKQEPGQGLEWIGYFNPYNHGTKYNEKFKGRATLTADKSSNTAYMDLSSLTSEDSAIYYCARSGPYAWFDTWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 321 bp, Artificial sequence
  Source 1..321: /note= “Description of artificial sequence Nucleotide sequence encoding a polypeptide of SEQ ID NO1”
gacattctgctgacccagtctccagccatcctgtctgtgagtccaggagaaagagtcagtttctcctgcagggccagtcagaacattggcacaagcatacagtggtatcaacaaagaacaaatggttctccaaggcttctcataaggtcttcttctgagtctatctctgggatcccttccaggtttagtggcagtggatcagggacagattttactcttagcatcaacagtgtggagtctgaagatattgcagattattactgtcaacaaagtaatacctggccattcacgttcggctcggggaccaagcttgaaatcaaa

SEQ ID NO: 6 - DNA, 354 bp, Artificial sequence
  Source 1..354: /note= “Description of artificial sequence Nucleotide sequence encoding a polypeptide of SEQ ID NO2”
gaggtgcagctgcagcagtcaggacctgaactggtaaagcctggggcttcagtgaagatgtcctgcaaggcctctggatacacattcactaattatattatccactgggtgaagcaggagcctggtcagggccttgaatggattggatattttaatccttacaatcatggtactaagtacaatgagaagttcaaaggcagggccacactaactgcagacaaatcctccaacacagcctacatggacctcagcagcctgacctctgaggactctgcgatctactactgtgcaagatcaggaccctatgcctggtttgacacctggggccaagggaccacggtcaccgtctcctca

SEQ ID NO: 7 - protein, 107 aa, Artificial sequence
  Source 1..107: /note= “Description of artificial sequence Part of amino acid sequence of humanised light chain designated humV2 (humV2 = VLm)”
DILLTQSPATLSLSPGERATFSCRASQNIGTSIQWYQQKTNGAPRLLIRSSSESISGIPSRFSGSGSGTDFTLTISSLEPEDFAVYYCQQSNTWPFTFGQGTKLEIK

SEQ ID NO: 8 - protein, 107 aa, Artificial sequence
  Source 1..107: /note= “Description of artificial sequence Part of amino acid sequence of humanised light chain designated humV1 (humV1 = VLh)”
DILLTQSPATLSLSPGERATLSCRASQNIGTSIQWYQQKPGQAPRLLIRSSSESISGIPSRFSGSGSGTDFTLTISSLEPEDFAVYYCQQSNTWPFTFGQGTKLEIK

SEQ ID NO: 9 - protein, 118 aa, Artificial sequence
  Source 1..118: /note= “Description of artificial sequence Part of amino acid sequence of humanised heavy chain designated VHE”
EVQLVESGAEVKKPGASVKVSCKASGYTFTNYIIHWVKQEPGQGLEWIGYFNPYNHGTKYNEKFKGRATLTANKSISTAYMELSSLRSEDTAVYYCARSGPYAWFDTWGQGTTVTVSS

SEQ ID NO: 10 - protein, 118 aa, Artificial sequence
  Source 1..118: /note= “Description of artificial sequence Part of amino acid sequence of humanised heavy chain designated VHQ”
QVQLVESGAEVKKPGASVKVSCKASGYTFTNYIIHWVKQEPGQGLEWIGYFNPYNHGTKYNEKFKGRATLTANKSISTAYMELSSLRSEDTAVYYCARSGPYAWFDTWGQGTTVTVSS

SEQ ID NO: 11 - DNA, 354 bp, Artificial sequence
  Source 1..354: /note= “Description of artificial sequence Nucleotide sequence encoding amino acid sequence SEQ ID NO9”
gaggtgcagctggtggagtcaggagccgaagtgaaaaagcctggggcttcagtgaaggtgtcctgcaaggcctctggatacacattcactaattatattatccactgggtgaagcaggagcctggtcagggccttgaatggattggatattttaatccttacaatcatggtactaagtacaatgagaagttcaaaggcagggccacactaactgcaaacaaatccatcagcacagcctacatggagctcagcagcctgcgctctgaggacactgcggtctactactgtgcaagatcaggaccctatgcctggtttgacacctggggccaagggaccacggtcaccgtctcctca

SEQ ID NO: 12 - DNA, 354 bp, Artificial sequence
  Source 1..354: /note= “Description of artificial sequence Nucleotide sequence encoding amino acid sequence SEQ ID NO10”
caggtgcagctggtggagtcaggagccgaagtgaaaaagcctggggcttcagtgaaggtgtcctgcaaggcctctggatacacattcactaattatattatccactgggtgaagcaggagcctggtcagggccttgaatggattggatattttaatccttacaatcatggtactaagtacaatgagaagttcaaaggcagggccacactaactgcaaacaaatccatcagcacagcctacatggagctcagcagcctgcgctctgaggacactgcggtctactactgtgcaagatcaggaccctatgcctggtttgacacctggggccaagggaccacggtcaccgtctcctca

SEQ ID NO: 13 - DNA, 321 bp, Artificial sequence
  Source 1..321: /note= “Description of artificial sequence Nucleotide sequence encoding amino acid sequence SEQ ID NO7”
gacattctgctgacccagtctccagccaccctgtctctgagtccaggagaaagagccactttctcctgcagggccagtcagaacattggcacaagcatacagtggtatcaacaaaaaacaaatggtgctccaaggcttctcataaggtcttcttctgagtctatctctgggatcccttccaggtttagtggcagtggatcagggacagattttactcttaccatcagcagtctggagcctgaagattttgcagtgtattactgtcaacaaagtaatacctggccattcacgttcggccaggggaccaagctggagatcaaa

SEQ ID NO: 14 - DNA, 321 bp, Artificial sequence
  Source 1..321: /note= “Description of artificial sequence Nucleotide sequence encoding amino acid sequence SEQ ID NO8”
gacattctgctgacccagtctccagccaccctgtctctgagtccaggagaaagagccactctctcctgcagggccagtcagaacattggcacaagcatacagtggtatcaacaaaaaccaggtcaggctccaaggcttctcataaggtcttcttctgagtctatctctgggatcccttccaggtttagtggcagtggatcagggacagattttactcttaccatcagcagtctggagcctgaagattttgcagtgtattactgtcaacaaagtaatacctggccattcacgttcggccaggggaccaagctggagatcaaa

SEQ ID NO: 15 - DNA, 8687 bp, Artificial sequence
  Source 1..8687: /note= “Description of artificial sequence Nucleotide sequence of the expression vector HCMV-G1 HuA6-VHQ (Complete DNA Sequence of a humanised heavy chain expression vector comprising SEQ ID NO12 (VHQ) from 3921-4274)”
agctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggttgctgactaattgagatgcatgctttgcatacttctgcctgctggggagcctggggactttccacaccctaactgacacacattccacagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcagcttggctgcagtgaataataaaatgtgtgtttgtccgaaatacgcgttttgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcaagcttgccgccaccatggactggacctggagggtgttctgcctgctggccgtggcccccggcgcccacagccaggtgcagctggtggagtcaggagccgaagtgaaaaagcctggggcttcagtgaaggtgtcctgcaaggcctctggatacacattcactaattatattatccactgggtgaagcaggagcctggtcagggccttgaatggattggatattttaatccttacaatcatggtactaagtacaatgagaagttcaaaggcagggccacactaactgcaaacaaatccatcagcacagcctacatggagctcagcagcctgcgctctgaggacactgcggtctactactgtgcaagatcaggaccctatgcctggtttgacacctggggccaagggaccacggtcaccgtctcctcaggtgagttctagaaggatcccaagctagctttctggggcaggccaggcctgaccttggctttggggcagggagggggctaaggtgaggcaggtggcgccagccaggtgcacacccaatgcccatgagcccagacactggacgctgaacctcgcggacagttaagaacccaggggcctctgcgccctgggcccagctctgtcccacaccgcggtcacatggcaccacctctcttgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagccaggctcagcgctcctgcctggacgcatcccggctatgcagccccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccggcaagcccccgctccccgggctctcgcggtcgcacgaggatgcttggcacgtaccccctgtacatacttcccgggcgcccagcatggaaataaagcacccagcgctgccctgggcccctgcgagactgtgatggttctttccacgggtcaggccgagtctgaggcctgagtggcatgagatctgatatcatcgatgaattcgagctcggtacccggggatcgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctagtcaaggcactatacatcaaatattccttattaacccctttacaaattaaaaagctaaaggtacacaatttttgagcatagttattaatagcagacactctatgcctgtgtggagtaagaaaaaacagtatgttatgattataactgttatgcctacttataaaggttacagaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagatccgggaccaaagcggccatcgtgcctccccactcctgcagttcgggggcatggatgcgcggatagccgctgctggtttcctggatgccgacggatttgcactgccggtagaactccgcgaggtcgtccagcctcaggcagcagctgaaccaactcgcgaggggatcgagcccggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagct

SEQ ID NO: 16 - DNA, 8687 bp, Artificial sequence
  Source 1..8687: /note= “Description of artificial sequence Nucleotide sequence of the expression vector HCMV-G1 HuA6-VHE (Complete DNA Sequence of a humanised heavy chain expression vector comprising SEQ ID NO 11 (VHE)from 3921-4274)”
agctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggttgctgactaattgagatgcatgctttgcatacttctgcctgctggggagcctggggactttccacaccctaactgacacacattccacagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcagcttggctgcagtgaataataaaatgtgtgtttgtccgaaatacgcgttttgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcaagcttgccgccaccatggactggacctggagggtgttctgcctgctggccgtggcccccggcgcccacagcgaggtgcagctggtggagtcaggagccgaagtgaaaaagcctggggcttcagtgaaggtgtcctgcaaggcctctggatacacattcactaattatattatccactgggtgaagcaggagcctggtcagggccttgaatggattggatattttaatccttacaatcatggtactaagtacaatgagaagttcaaaggcagggccacactaactgcaaacaaatccatcagcacagcctacatggagctcagcagcctgcgctctgaggacactgcggtctactactgtgcaagatcaggaccctatgcctggtttgacacctggggccaagggaccacggtcaccgtctcctcaggtgagttctagaaggatcccaagctagctttctggggcaggccaggcctgaccttggctttggggcagggagggggctaaggtgaggcaggtggcgccagccaggtgcacacccaatgcccatgagcccagacactggacgctgaacctcgcggacagttaagaacccaggggcctctgcgccctgggcccagctctgtcccacaccgcggtcacatggcaccacctctcttgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagccaggctcagcgctcctgcctggacgcatcccggctatgcagccccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccggcaagcccccgctccccgggctctcgcggtcgcacgaggatgcttggcacgtaccccctgtacatacttcccgggcgcccagcatggaaataaagcacccagcgctgccctgggcccctgcgagactgtgatggttctttccacgggtcaggccgagtctgaggcctgagtggcatgagatctgatatcatcgatgaattcgagctcggtacccggggatcgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctagtcaaggcactatacatcaaatattccttattaacccctttacaaattaaaaagctaaaggtacacaatttttgagcatagttattaatagcagacactctatgcctgtgtggagtaagaaaaaacagtatgttatgattataactgttatgcctacttataaaggttacagaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagatccgggaccaaagcggccatcgtgcctccccactcctgcagttcgggggcatggatgcgcggatagccgctgctggtttcctggatgccgacggatttgcactgccggtagaactccgcgaggtcgtccagcctcaggcagcagctgaaccaactcgcgaggggatcgagcccggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagct

SEQ ID NO: 17 - DNA, 9400 bp, Artificial sequence
  Source 1..9400: /note= “Description of artificial sequence Nucleotide sequence of the expression vector HCMV-K HuAb-VL1 hum V1 (Complete DNA Sequence of a humanised light chain expression vector comprising SEQ ID NO 14 (humV1=VLh) from 3964-4284)
ctagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggttgctgactaattgagatgcatgctttgcatacttctgcctgctggggagcctggggactttccacaccctaactgacacacattccacagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcagcttggctgcagtgaataataaaatgtgtgtttgtccgaaatacgcgttttgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcaagcttgatatcgaattcctgcagcccgggggatccgcccgcttgccgccaccatggagacccccgcccagctgctgttcctgctgctgctgtggctgcccgacaccaccggcgacattctgctgacccagtctccagccaccctgtctctgagtccaggagaaagagccactctctcctgcagggccagtcagaacattggcacaagcatacagtggtatcaacaaaaaccaggtcaggctccaaggcttctcataaggtcttcttctgagtctatctctgggatcccttccaggtttagtggcagtggatcagggacagattttactcttaccatcagcagtctggagcctgaagattttgcagtgtattactgtcaacaaagtaatacctggccattcacgttcggccaggggaccaagctggagatcaaacgtgagtattctagaaagatcctagaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagaagtgcccccacctgctcctcagttccagcctgaccccctcccatcctttggcctctgaccctttttccacaggggacctacccctattgcggtcctccagctcatctttcacctcacccccctcctcctccttggctttaattatgctaatgttggaggagaatgaataaataaagtgaatctttgcacctgtggtttctctctttcctcatttaataattattatctgttgtttaccaactactcaatttctcttataagggactaaatatgtagtcatcctaaggcgcataaccatttataaaaatcatccttcattctattttaccctatcatcctctgcaagacagtcctccctcaaacccacaagccttctgtcctcacagtcccctgggccatggtaggagagacttgcttccttgttttcccctcctcagcaagccctcatagtcctttttaagggtgacaggtcttacagtcatatatcctttgattcaattccctgagaatcaaccaaagcaaatttttcaaaagaagaaacctgctataaagagaatcattcattgcaacatgatataaaataacaacacaataaaagcaattaaataaacaaacaatagggaaatgtttaagttcatcatggtacttagacttaatggaatgtcatgccttatttacatttttaaacaggtactgagggactcctgtctgccaagggccgtattgagtactttccacaacctaatttaatccacactatactgtgagattaaaaacattcattaaaatgttgcaaaggttctataaagctgagagacaaatatattctataactcagcaatcccacttctagatgactgagtgtccccacccaccaaaaaactatgcaagaatgttcaaagcagctttatttacaaaagccaaaaattggaaatagcccgattgtccaacaatagaatgagttattaaactgtggtatgtttatacattagaatacccaatgaggagaattaacaagctacaactatacctactcacacagatgaatctcataaaaataatgttacataagagaaactcaatgcaaaagatatgttctgtatgttttcatccatataaagttcaaaaccaggtaaaaataaagttagaaatttggatggaaattactcttagctgggggtgggcgagttagtgcctgggagaagacaagaaggggcttctggggtcttggtaatgttctgttcctcgtgtggggttgtgcagttatgatctgtgcactgttctgtatacacattatgcttcaaaataacttcacataaagaacatcttatacccagttaatagatagaagaggaataagtaataggtcaagaccacgcagctggtaagtgggggggcctgggatcaaatagctacctgcctaatcctgccctcttgagccctgaatgagtctgccttccagggctcaaggtgctcaacaaaacaacaggcctgctattttcctggcatctgtgccctgtttggctagctaggagcacacatacatagaaattaaatgaaacagaccttcagcaaggggacagaggacagaattaaccttgcccagacactggaaacccatgtatgaacactcacatgtttgggaagggggaagggcacatgtaaatgaggactcttcctcattctatggggcactctggccctgcccctctcagctactcatccatccaacacacctttctaagtacctctctctgcctacactctgaaggggttcaggagtaactaacacagcatcccttccctcaaatgactgacaatccctttgtcctgctttgtttttctttccagtcagtactgggaaagtggggaaggacagtcatggagaaactacataaggaagcaccttgcccttctgcctcttgagaatgttgatgagtatcaaatctttcaaactttggaggtttgagtaggggtgagactcagtaatgtcccttccaatgacatgaacttgctcactcatccctgggggccaaattgaacaatcaaaggcaggcataatccagctatgaattctaggatcgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctagtcaaggcactatacatcaaatattccttattaacccctttacaaattaaaaagctaaaggtacacaatttttgagcatagttattaatagcagacactctatgcctgtgtggagtaagaaaaaacagtatgttatgattataactgttatgcctacttataaaggttacagaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagatccgggaccaaagcggccatcgtgcctccccactcctgcagttcgggggcatggatgcgcggatagccgctgctggtttcctggatgccgacggatttgcactgccggtagaactccgcgaggtcgtccagcctcaggcagcagctgaaccaactcgcgaggggatcgagcccggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaag

SEQ ID NO: 18 - DNA, 9362 bp, Artificial sequence
  Source 1..9362: /note= ”Description of artificial sequence Nucleotide sequence of the expression vector HCMV-K HuAb-VL1 hum V2 (Complete DNA Sequence of a humanised light chain expression vector comprising SEQ ID NO 13 (humV2=VLm) from 3926-4246)“
ctagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggttgctgactaattgagatgcatgctttgcatacttctgcctgctggggagcctggggactttccacaccctaactgacacacattccacagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcagcttggctgcagtgaataataaaatgtgtgtttgtccgaaatacgcgttttgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcaagcttgccgccaccatggagacccccgcccagctgctgttcctgctgctgctgtggctgcccgacaccaccggcgacattctgctgacccagtctccagccaccctgtctctgagtccaggagaaagagccactttctcctgcagggccagtcagaacattggcacaagcatacagtggtatcaacaaaaaacaaatggtgctccaaggcttctcataaggtcttcttctgagtctatctctgggatcccttccaggtttagtggcagtggatcagggacagattttactcttaccatcagcagtctggagcctgaagattttgcagtgtattactgtcaacaaagtaatacctggccattcacgttcggccaggggaccaagctggagatcaaacgtgagtattctagaaagatcctagaattctaaactctgagggggtcggatgacgtggccattctttgcctaaagcattgagtttactgcaaggtcagaaaagcatgcaaagccctcagaatggctgcaaagagctccaacaaaacaatttagaactttattaaggaatagggggaagctaggaagaaactcaaaacatcaagattttaaatacgcttcttggtctccttgctataattatctgggataagcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagaagtgcccccacctgctcctcagttccagcctgaccccctcccatcctttggcctctgaccctttttccacaggggacctacccctattgcggtcctccagctcatctttcacctcacccccctcctcctccttggctttaattatgctaatgttggaggagaatgaataaataaagtgaatctttgcacctgtggtttctctctttcctcatttaataattattatctgttgtttaccaactactcaatttctcttataagggactaaatatgtagtcatcctaaggcgcataaccatttataaaaatcatccttcattctattttaccctatcatcctctgcaagacagtcctccctcaaacccacaagccttctgtcctcacagtcccctgggccatggtaggagagacttgcttccttgttttcccctcctcagcaagccctcatagtcctttttaagggtgacaggtcttacagtcatatatcctttgattcaattccctgagaatcaaccaaagcaaatttttcaaaagaagaaacctgctataaagagaatcattcattgcaacatgatataaaataacaacacaataaaagcaattaaataaacaaacaatagggaaatgtttaagttcatcatggtacttagacttaatggaatgtcatgccttatttacatttttaaacaggtactgagggactcctgtctgccaagggccgtattgagtactttccacaacctaatttaatccacactatactgtgagattaaaaacattcattaaaatgttgcaaaggttctataaagctgagagacaaatatattctataactcagcaatcccacttctagatgactgagtgtccccacccaccaaaaaactatgcaagaatgttcaaagcagctttatttacaaaagccaaaaattggaaatagcccgattgtccaacaatagaatgagttattaaactgtggtatgtttatacattagaatacccaatgaggagaattaacaagctacaactatacctactcacacagatgaatctcataaaaataatgttacataagagaaactcaatgcaaaagatatgttctgtatgttttcatccatataaagttcaaaaccaggtaaaaataaagttagaaatttggatggaaattactcttagctgggggtgggcgagttagtgcctgggagaagacaagaaggggcttctggggtcttggtaatgttctgttcctcgtgtggggttgtgcagttatgatctgtgcactgttctgtatacacattatgcttcaaaataacttcacataaagaacatcttatacccagttaatagatagaagaggaataagtaataggtcaagaccacgcagctggtaagtgggggggcctgggatcaaatagctacctgcctaatcctgccctcttgagccctgaatgagtctgccttccagggctcaaggtgctcaacaaaacaacaggcctgctattttcctggcatctgtgccctgtttggctagctaggagcacacatacatagaaattaaatgaaacagaccttcagcaaggggacagaggacagaattaaccttgcccagacactggaaacccatgtatgaacactcacatgtttgggaagggggaagggcacatgtaaatgaggactcttcctcattctatggggcactctggccctgcccctctcagctactcatccatccaacacacctttctaagtacctctctctgcctacactctgaaggggttcaggagtaactaacacagcatcccttccctcaaatgactgacaatccctttgtcctgctttgtttttctttccagtcagtactgggaaagtggggaaggacagtcatggagaaactacataaggaagcaccttgcccttctgcctcttgagaatgttgatgagtatcaaatctttcaaactttggaggtttgagtaggggtgagactcagtaatgtcccttccaatgacatgaacttgctcactcatccctgggggccaaattgaacaatcaaaggcaggcataatccagctatgaattctaggatcgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctagtcaaggcactatacatcaaatattccttattaacccctttacaaattaaaaagctaaaggtacacaatttttgagcatagttattaatagcagacactctatgcctgtgtggagtaagaaaaaacagtatgttatgattataactgttatgcctacttataaaggttacagaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagatccgggaccaaagcggccatcgtgcctccccactcctgcagttcgggggcatggatgcgcggatagccgctgctggtttcctggatgccgacggatttgcactgccggtagaactccgcgaggtcgtccagcctcaggcagcagctgaaccaactcgcgaggggatcgagcccggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaag

SEQ ID NO: 19 - protein, 11 aa, Artificial sequence
  Source 1..11: /note= ”Description of artificial sequence Hypervariable region CDR1prime in the CD45RO/RB binding molecule of SEQ ID NO 1“
RASQNIGTSIQ

SEQ ID NO: 20 - protein, 7 aa, Artificial sequence
  Source 1..7: /note= ”Description of artificial sequence Hypervariable region CDR2prime in the CD45RO/RB binding molecule of SEQ ID NO 1“
SSSESIS

SEQ ID NO: 21 - protein, 9 aa, Artificial sequence
  Source 1..9: /note= ”Description of artificial sequence Hypervariable region CDR3prime in the CD45RO/RB binding molecule of SEQ ID NO 1“
QQSNTWPFT

SEQ ID NO: 22 - protein, 5 aa, Artificial sequence
  Source 1..5: /note= ”Description of artificial sequence Hypervariable region CDR1 in a CD45RO/RB binding molecule of SEQ ID NO 2“
NYIIH

SEQ ID NO: 23 - protein, 17 aa, Artificial sequence
  Source 1..17: /note= ”Description of artificial sequence Hypervariable region CDR2 in a CD45RO/RB binding molecule of SEQ ID NO 2“
YFNPYNHGTKYNEKFKG

SEQ ID NO: 24 - protein, 9 aa, Artificial sequence
  Source 1..9: /note= ”Description of artificial sequence Hypervariable region CDR3 in a CD45RO/RB binding molecule of SEQ ID NO 2“
SGPYAWFDT

SEQ ID NO: 25 - DNA, 33 bp, Artificial sequence
  Source 1..33: /note= ”Description of artificial sequence Polynucleotide sequence encoding the amino acid sequence of CDR1“
ggccagtcagaacattggcacaagcatacagtg

SEQ ID NO: 26 - DNA, 21 bp, Artificial sequence
  Source 1..21: /note= ”Description of artificial sequence Polynucleotide sequence encoding the amino acid sequence of CDR2“
ttcttctgagtctatctctgg

SEQ ID NO: 27 - DNA, 27 bp, Artificial sequence
  Source 1..27: /note= ”Description of artificial sequence Polynucleotide sequence encoding the amino acid sequence of CDR3“
acaaagtaatacctggccattcacgtt

SEQ ID NO: 28 - DNA, 15 bp, Artificial sequence
  Source 1..15: /note= ”Description of artificial sequence Polynucleotide sequence encoding the amino acid sequence of CDR1prime“
ttatattatccactg

SEQ ID NO: 29 - DNA, 51 bp, Artificial sequence
  Source 1..51: /note= ”Description of artificial sequence Polynucleotide sequence encoding the amino acid sequence of CDR2prime“
ttttaatccttacaatcatggtactaagtacaatgagaagttcaaaggcag

SEQ ID NO: 30 - DNA, 27 bp, Artificial sequence
  Source 1..27: /note= ”Description of artificial sequence Polynucleotide sequence encoding the amino acid sequence of CDR3prime“
aggaccctatgcctggtttgacacctg

SEQ ID NO: 31 - protein, 118 aa, Artificial sequence
  Source 1..118: /note= ”Description of artificial sequence Part of amino acid sequence of humanised heavy chain designated VHE-N73D“
EVQLVESGAEVKKPGASVKVSCKASGYTFTNYIIHWVKQEPGQGLEWIGYFNPYNHGTKYNEKFKGRATLTADKSISTAYMELSSLRSEDTAVYYCARSGPYAWFDTWGQGTTVTVSS

SEQ ID NO: 32 - protein, 118 aa, Artificial sequence
  Source 1..118: /note= ”Description of artificial sequence Part of amino acid sequence of humanised heavy chain designated VHQ-N73D“
QVQLVESGAEVKKPGASVKVSCKASGYTFTNYIIHWVKQEPGQGLEWIGYFNPYNHGTKYNEKFKGRATLTADKSISTAYMELSSLRSEDTAVYYCARSGPYAWFDTWGQGTTVTVSS

SEQ ID NO: 33 - DNA, 321 bp, Artificial sequence
  Source 1..321: /note= ”Description of artificial sequence Nucleotide sequence encoding amino acid sequence SEQ ID NO 8“
gacattctgctgacccagtctccagccaccctgtctctgagtccaggagaaagagccactctctcctgcagggccagtcagaacattggcacaagcatacagtggtatcaacaaaaaccaggtcaggctccaaggcttctcataaggtcttcttctgagtctatctctgggatcccttccaggtttagtggcagtggatcagggacagattttactcttaccatcagcagtctggagcctgaagattttgcagtgtattactgtcaacaaagtaatacctggccattcacgttcggccaggggaccaagcttgaaatcaaa

SEQ ID NO: 34 - DNA, 354 bp, Artificial sequence
  Source 1..354: /note= ”Description of artificial sequence Nucleotide sequence encoding amino acid sequence SEQ ID NO 31“
gaggtgcagctggtggagtcaggagccgaagtgaaaaagcctggggcttcagtgaaggtgtcctgcaaggcctctggatacacattcactaattatattatccactgggtgaagcaggagcctggtcagggccttgaatggattggatattttaatccttacaatcatggtactaagtacaatgagaagttcaaaggcagggccacactaactgcagacaaatccatcagcacagcctacatggagctcagcagcctgcgctctgaggacactgcggtctactactgtgcaagatcaggaccctatgcctggtttgacacctggggccaagggaccacggtcaccgtctcctca

SEQ ID NO: 35 - DNA, 354 bp, Artificial sequence
  Source 1..354: /note= ”Description of artificial sequence Nucleotide sequence encoding amino acid sequence SEQ ID NO 32“
caggtgcagctggtggagtcaggagccgaagtgaaaaagcctggggcttcagtgaaggtgtcctgcaaggcctctggatacacattcactaattatattatccactgggtgaagcaggagcctggtcagggccttgaatggattggatattttaatccttacaatcatggtactaagtacaatgagaagttcaaaggcagggccacactaactgcagacaaatccatcagcacagcctacatggagctcagcagcctgcgctctgaggacactgcggtctactactgtgcaagatcaggaccctatgcctggtttgacacctggggccaagggaccacggtcaccgtctcctca

SEQ ID NO: 36 - DNA, 8096 bp, Artificial sequence
  Source 1..8096: /note= ”Description of artificial sequence Nucleotide sequence of expression vector LCVL1Sp20“
ctagagtcctagagaggtctggtggagcctgcaaaagtccagctttcaaaggaacacagaagtatgtgtatggaatattagaagatgttgcttttactcttaagttggttcctaggaaaaatagttaaatactgtgactttaaaatgtgagagggttttcaagtactcatttttttaaatgtccaaaatttttgtcaatcaatttgaggtcttgtttgtgtagaactgacattacttaaagtttaaccgaggaatgggagtgaggctctctcataccctattcagaactgacttttaacaataataaattaagtttaaaatatttttaaatgaattgagcaatgttgagttggagtcaagatggccgatcagaaccagaacacctgcagcagctggcaggaagcaggtcatgtggcaaggctatttggggaagggaaaataaaaccactaggtaaacttgtagctgtggtttgaagaagtggttttgaaacactctgtccagccccaccaaaccgaaagtccaggctgagcaaaacaccacctgggtaatttgcatttctaaaataagttgaggattcagccgaaactggagaggtcctcttttaacttattgagttcaaccttttaattttagcttgagtagttctagtttccccaaacttaagtttatcgacttctaaaatgtatttagaactcattttcaaaattaggttatgtaagaaattgaaggactttagtgtctttaatttctaatatatttagaaaacttcttaaaattactctattattcttccctctgattattggtctccattcaattcttttccaatacccgaagcatttacagtgactttgttcatgatcttttttagttgtttgttttgccttactattaagactttgacattctggtcaaaacggcttcacaaatctttttcaagaccactttctgagtattcattttaggagaaatactttttttttaaatgaatgcaattatctaggacctgcaggcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagaagtgcccccacctgctcctcagttccagcctgaccccctcccatcctttggcctctgaccctttttccacaggggacctacccctattgcggtcctccagctcatctttcacctcacccccctcctcctccttggctttaattatgctaatgttggaggagaatgaataaataaagtgaatctttgcacctgtggtttctctctttcctcatttaataattattatctgttgttttaccaactactcaatttctcttataagggactaaatatgtagtcatcctaaggcgggatatcgagatctgaagctgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctagtcaaggcactatacatcaaatattccttattaacccctttacaaattaaaaagctaaaggtacacaatttttgagcatagttattaatagcagacactctatgcctgtgtggagtaagaaaaaacagtatgttatgattataactgttatgcctacttataaaggttacagaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagatccggaccaaagcggccatcgtgcctccccactcctgcagttcgggggcatggatgcgcggatagccgctgctggtttcctggatgccgacggatttgcactgccggtagaactccgcgaggtcgtccagcctcaggcagcagctgaaccaactcgcgaggggatcgagcatcccccatggtcttataaaaatgcatagctttaggaggggagcagagaacttgaaagcatcttcctgttagtctttcttctcgtagacttcaaacttatacttgatgcctttttcctcctggacctcagagaggacgcctgggtattctgggagaagtttatatttccccaaatcaatttctgggaaaaacgtgtcactttcaaattcctgcatgatccttgtcacaaagagtctgaggtggcctggttgattcatggcttcctggtaaacagaactgcctccgactatccaaaccatgtctactttacttgccaattccggttgttcaataagtcttaaggcatcatccaaacttttggcaagaaaatgagctcctcgtggtggttctttgagttctctactgagaactatattaattctgtcctttaaaggtcgattcttctcaggaatggagaaccaggttttcctacccataatcaccagattctgtttaccttccactgaagaggttgtggtcattctttggaagtacttgaactcgttcctgagcggaggccagggtcggtctccgttcttgccaatccccatattttgggacacggcgacgatgcagttcaatggtcgaaccatgatggcagcggggataaaatcctaccagccttcacgctaggattgccgtcaagtttgggggtaccgagctcgaattagctttttgcaaaagcctaggcctccaaaaaagcctcctcactacttctggaatagctcagagggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggccgcatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcagctgctcgaggaagagctcaaacccatgctactctctggcttgatggaagcaacgctttcatagctgagctgtcataaataataaagagatttttttattaatattgaaaagatgggttatttatgtaagactctgtcttcattttaaaaaccacaccttccagtagtattctgttactgttctggcaatcactgtgatcaagaagctacacggtgagttgtgcttctcagtcctaagggatacatctacaagaggctcccatactcgaagctcaggaaacattgtagaaaaggaggcaaaagactgacagagccagaggaccaagaaatttgttgtgaggttgtgtctcctactaacaatataagcaatatctataaattgttgatatcatggctactaaaatgtgagttgaacgaggaggacacaaatgaacatgacaatcagaatgaggcctctcacctgcaaaaaacactatagagaagcagataaagctgtcagcagaagaggcgcacctccttatagaagaagcctaccaggtttgatatatcagccttgaaaacctacatagtatttacattatatcgagtctatgagacatatttagtaatgcatatgtatgtgtgtgtgtgcatgtatgtgtgtaaatacatatgttcatagaaaaatgtgtaaaaagagatcatgaatttaagagagaactgggacaatttttttcagggagttgtaatcaggaaagttaagggaaaaatgttgtaattaaaattcaggctcagaaacaaacaaaggaaaagaaaaaaaaacaacaacaacaacaaaaaaacaaaacaaaggagaagctgtatggccacaatagcatctacagctaactgtgaaaggataatggaacaagttatgtactgcctagagcagtatgatgcctaaatcatctcgacatggaggaaaatagaacaaagacactctacatagactatgatagaaatcaaaataaggtgtaagacatagaacattagttttgtttgttgttcaaagagactcacattcccacaaaaaaatctgtgggattttacaggtctgcaataagctgctgacctgatgatttctgcagctgtgcctaccctttgctgatttgcatgtacccaaagcatagcttactgacatgaggatttcttcatagtcaggtcacaccctttgctggagtcagaatcacactgatcacacacagtcatgagtgtgctcactcaggtcctggcgttgctgctgctgtggcttacaggtaatgaagacagcactagaattttattgagcttcctgtacactgtgctgcttgtctctgtgaaaattctcttgtgaattaatcatgtggggatctgttttcaatttttcaattgtaggtacgcgttgtgacattctgctgacccagtctccagccaccctgtctctgagtccaggagaaagagccactctctcctgcagggccagtcagaacattggcacaagcatacagtggtatcaacaaaaaccaggtcaggctccaaggcttctcataaggtcttcttctgagtctatctctgggatcccttccaggtttagtggcagtggatcagggacagattttactcttaccatcagcagtctggagcctgaagattttgcagtgtattactgtcaacaaagtaatacctggccattcacgttcggccaggggaccaagcttgaaatcaaacgtaagtagaatccaaagtctctttcttccgttgtctatgtctgtggcttctatgtctaaaaatgatgtataaaatcttactctgaaaccagattctggcactctccaaggcaaagatacagagtaactccgtaagcaaagctgggaataggctagacatgttctctggagaatgaatgccagtgtaataattaacacaagtgatagtttcagaaatgctctagtt

SEQ ID NO: 37 - DNA, 11563 bp, Artificial sequence
  Source 1..11563: /note= ”Description of artificial sequence Nucleotide sequence of the expression vector HCVHEN73DSp20“
ctagagaggtctggtggagcctgcaaaagtccagctttcaaaggaacacagaagtatgtgtatggaatattagaagatgttgcttttactcttaagttggttcctaggaaaaatagttaaatactgtgactttaaaatgtgagagggttttcaagtactcatttttttaaatgtccaaaatttttgtcaatcaatttgaggtcttgtttgtgtagaactgacattacttaaagtttaaccgaggaatgggagtgaggctctctcataccctattcagaactgacttttaacaataataaattaagtttaaaatatttttaaatgaattgagcaatgttgagttggagtcaagatggccgatcagaaccagaacacctgcagcagctggcaggaagcaggtcatgtggcaaggctatttggggaagggaaaataaaaccactaggtaaacttgtagctgtggtttgaagaagtggttttgaaacactctgtccagccccaccaaaccgaaagtccaggctgagcaaaacaccacctgggtaatttgcatttctaaaataagttgaggattcagccgaaactggagaggtcctcttttaacttattgagttcaaccttttaattttagcttgagtagttctagtttccccaaacttaagtttatcgacttctaaaatgtatttaagctttctggggcaggccaggcctgaccttggctttggggcagggagggggctaaggtgaggcaggtggcgccagccaggtgcacacccaatgcccatgagcccagacactggacgctgaacctcgcggacagttaagaacccaggggcctctgcgccctgggcccagctctgtcccacaccgcggtcacatggcaccacctctcttgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttggtgagaggccagcacagggagggagggtgtctgctggaagccaggctcagcgctcctgcctggacgcatcccggctatgcagtcccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatgagtgcgacggccggcaagcccccgctccccgggctctcgcggtcgcacgaggatgcttggcacgtaccccgtctacatacttcccaggcacccagcatggaaataaagcacccaccactgccctgggcccctgcgagactgtgatggttctttccacgggtcaggccgagtctgaggcctgagtggcatgagggaggcagagcgggtcccactgtccccacactggcccaggctgtgcaggtgtgcctgggccgcctagggtggggctcagccaggggctgccctcggcagggtgggggatttgccagcgtggccctccctccagcagcagctgccctgggctgggccacgagaagccctaggagcccctggggacagacacacagcccctgcctctgtaggagactgtcctgtcctgtgagcgccctgtcctccgaccccgatgcccactcgggggcatgcctagtccatgcgcgtagggacaggccctccctcacccatctacccccacggcactaacccctggcagccctgcccagcctcgcacccgcatggggacacaaccgactccggggacatgcactctcgggccctgtggagggactggtgcagatgcccacacacacactcagcccagacccgttcaacaaaccccgcactgaggttggtcgagcgggagtgcggccagagcctgcctcggccgtcagggaggactcccgggctcactcgaaggaggtgccaccatttcagctttggtagcttttcttcttcttttaaattttctaaagctcattaattgtctttgatgtttcttttgtgatgacaataaaatatcctttttaagtcttgtacttcgtgatgggagccgccttcctgtgtccacgcgcctcctgcccccggtgggaagcacggtcaggaggaggctggtccagctgcacctcgggggctccctgcactcgccccccgcctcctgcagccacacgcattgcccgagcgaccctccctggcccctgtcactacatggacccctggggcttctcctcttttctacatggatgcagtttctcctcctgctgggcacggtgctgcctgccctggtcactctgcgggggacagggcctccagggaaagctgggtcgaggctgggagctggctcaggctggccaggcagagccacagggagggccttccagaaccaaccatggtccgaagcgagaggtgggtgtcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctagtcaaggcactatacatcaaatattccttattaacccctttacaaattaaaaagctaaaggtacacaatttttgagcatagttattaatagcagacactctatgcctgtgtggagtaagaaaaaacagtatgttatgattataactgttatgcctacttataaaggttacagaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagatccggaccaaagcggccatcgtgcctccccactcctgcagttcgggggcatggatgcgcggatagccgctgctggtttcctggatgccgacggatttgcactgccggtagaactccgcgaggtcgtccagcctcaggcagcagctgaaccaactcgcgaggggatcgagcccggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcgagctcggtacccatcagccaaaaagcatgcctgccacacaacatcaatttctggaaaacgctacacttaattatttctagtagaacagctctttggtttgccaaaaagaatcacctatagtggcatctaagcacaaaaaggagaaaaaaatcacaaagaaatgattgagaggcataataaaaattatcaaaaaattatgagttttacgatttcatctttttccaagttgaaatcatagggtggctttaacacagtgacaaggaatgtgcatgctgccattatggtgctctgcctaaaatggttggagccttgtcatgctacagagaaactgtcatacagcagggggtgccaaatttccatatttttttatatcattgagcaggtgcacagaagaccagaaagcactttctatcaggctggccttcctcttcctttccagtatgaagcaaaaactgccaatgaaactagcaattgttaaattcctttttcaaacagtatttgtgctatcagaacatagtgcattcaaaagtctagcctgagagaacaacccagttttattcattcctcctactacctctctcattcccactgtttgtgttctccctcccattttaattgtctatctagtccaaactaagcacacgatccagtccacattaaacaacatgttttcactttaagtcaaatacaagacacctttaatatcagcccttgttcataatcgtgcttctagtgacttaatgtacatgtcacactgtactgttgggttttgtgtctcatcatgaacaatgttgtgaaggtattaagtggagagtaagcagaattagattcctctaatgatgcacacccacactaagagcagaaataatattaaaaatagaaaaaaaagttttacatgagatttcaaatacccaggtatgagctgcagtttcttcaagttaaagcatcgaggttgtcagttacactattacaggaaacatatgcagagtttttattttagtatattagttttcacatatgtggaattactattaaactattctttcttttcaaatgcttaccattgtaaatgagtttgtgactttgtgtaggtgagtgcacatgactctggatgcctaagaggactgaagaagttggagttataggtagttttattctacttgactgttcagtgctaaaaatacaactgaggtcctttaaactgctgttcatgaacttcttaattgatatatctcatgagatctctaaactatttttattatgacacgtttcaccattttcactgtaacgatttttatgttttatattaatgtaactatatgacacttcccaaaatccccatattcacaattgaactgtttcaaagttttaccttgacttatgggaaatgaaaacccacattttataattttaaaatgaaatgtttattttatatttctgcaaatttcacaaggaaagattagtcactggtgtgtgagagcagaggagcataagagttcaggaatagaatccattatgattctggaggcaaggaagaactgatgccaaggtttcagtataagagcagtatccactggaaaggataaagtcactacatctgagcacagagcaggacatctacataatgagtggtcactaatgggccactgttacactgttatatgtataaggctcaagaatgagcactgaggctgtaaggtgtatgggtgaggacatcaggatgtaaacccagctcaggtagaggactcagaggacagcacagtcagcatgaactaataaacatcagataagataaggcacaagctcagctatatagggtaagggatctttgtaaatctgattgtgcatccagtctagttcaatgtgacttaggaagcccagtcatatgcaaatctagagaagactttagagtagaaatctgaggctcacctcacataccagcaagcgagtgaccagttagtcttaaggcaccacttcttagacatcatggcttgggtgtggaccttgccattcctgatggcagctgcccaaagtaagacatcagaaaaaagagttccaaggggaattgaagcagttccatgaatactcaccttcctgtgttcttttcacaggtgtccaggcagaggtgcagctggtggagtcaggagccgaagtgaaaaagcctggggcttcagtgaaggtgtcctgcaaggcctctggatacacattcactaattatattatccactgggtgaagcaggagcctggtcagggccttgaatggattggatattttaatccttacaatcatggtactaagtacaatgagaagttcaaaggcagggccacactaactgcagacaaatccatcagcacagcctacatggagctcagcagcctgcgctctgaggacactgcggtctactactgtgcaagatcaggaccctatgcctggtttgacacctggggccaagggaccacggtcaccgtctcctcaggtaagaatggccactctagggcctttgttttctgctgctgcctgtgggatttcatgagcattgcaaagttgtcctcgggacatgttccgaggggacctgggcggactggccaggaggggacgggcactggggtgccttgaggatctgggagcctctgtggattttccgatgcctttggaaaatgggactgaggttgggtgcgtctgagacagtaactcagcctgggggcttggtgaagatcgccgcacagcagcgagtccgtgaaatatcttatttagacttgtgaggtgcgctgtgtgtcaatttacatcttaaatcctttattggctggaaagagaattgttggagtgggtgaatccagccaggagggacgcggggggatcca

SEQ ID NO: 38 - DNA, 11563 bp, Artificial sequence
  Source 1..11563: /note= ”Description of artificial sequence Nucleotide sequence of the expression vector HCVHQN73DSp20“
ctagagaggtctggtggagcctgcaaaagtccagctttcaaaggaacacagaagtatgtgtatggaatattagaagatgttgcttttactcttaagttggttcctaggaaaaatagttaaatactgtgactttaaaatgtgagagggttttcaagtactcatttttttaaatgtccaaaatttttgtcaatcaatttgaggtcttgtttgtgtagaactgacattacttaaagtttaaccgaggaatgggagtgaggctctctcataccctattcagaactgacttttaacaataataaattaagtttaaaatatttttaaatgaattgagcaatgttgagttggagtcaagatggccgatcagaaccagaacacctgcagcagctggcaggaagcaggtcatgtggcaaggctatttggggaagggaaaataaaaccactaggtaaacttgtagctgtggtttgaagaagtggttttgaaacactctgtccagccccaccaaaccgaaagtccaggctgagcaaaacaccacctgggtaatttgcatttctaaaataagttgaggattcagccgaaactggagaggtcctcttttaacttattgagttcaaccttttaattttagcttgagtagttctagtttccccaaacttaagtttatcgacttctaaaatgtatttaagctttctggggcaggccaggcctgaccttggctttggggcagggagggggctaaggtgaggcaggtggcgccagccaggtgcacacccaatgcccatgagcccagacactggacgctgaacctcgcggacagttaagaacccaggggcctctgcgccctgggcccagctctgtcccacaccgcggtcacatggcaccacctctcttgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttggtgagaggccagcacagggagggagggtgtctgctggaagccaggctcagcgctcctgcctggacgcatcccggctatgcagtcccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatgagtgcgacggccggcaagcccccgctccccgggctctcgcggtcgcacgaggatgcttggcacgtaccccgtctacatacttcccaggcacccagcatggaaataaagcacccaccactgccctgggcccctgcgagactgtgatggttctttccacgggtcaggccgagtctgaggcctgagtggcatgagggaggcagagcgggtcccactgtccccacactggcccaggctgtgcaggtgtgcctgggccgcctagggtggggctcagccaggggctgccctcggcagggtgggggatttgccagcgtggccctccctccagcagcagctgccctgggctgggccacgagaagccctaggagcccctggggacagacacacagcccctgcctctgtaggagactgtcctgtcctgtgagcgccctgtcctccgaccccgatgcccactcgggggcatgcctagtccatgcgcgtagggacaggccctccctcacccatctacccccacggcactaacccctggcagccctgcccagcctcgcacccgcatggggacacaaccgactccggggacatgcactctcgggccctgtggagggactggtgcagatgcccacacacacactcagcccagacccgttcaacaaaccccgcactgaggttggtcgagcgggagtgcggccagagcctgcctcggccgtcagggaggactcccgggctcactcgaaggaggtgccaccatttcagctttggtagcttttcttcttcttttaaattttctaaagctcattaattgtctttgatgtttcttttgtgatgacaataaaatatcctttttaagtcttgtacttcgtgatgggagccgccttcctgtgtccacgcgcctcctgcccccggtgggaagcacggtcaggaggaggctggtccagctgcacctcgggggctccctgcactcgccccccgcctcctgcagccacacgcattgcccgagcgaccctccctggcccctgtcactacatggacccctggggcttctcctcttttctacatggatgcagtttctcctcctgctgggcacggtgctgcctgccctggtcactctgcgggggacagggcctccagggaaagctgggtcgaggctgggagctggctcaggctggccaggcagagccacagggagggccttccagaaccaaccatggtccgaagcgagaggtgggtgtcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctagtcaaggcactatacatcaaatattccttattaacccctttacaaattaaaaagctaaaggtacacaatttttgagcatagttattaatagcagacactctatgcctgtgtggagtaagaaaaaacagtatgttatgattataactgttatgcctacttataaaggttacagaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagatccggaccaaagcggccatcgtgcctccccactcctgcagttcgggggcatggatgcgcggatagccgctgctggtttcctggatgccgacggatttgcactgccggtagaactccgcgaggtcgtccagcctcaggcagcagctgaaccaactcgcgaggggatcgagcccggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcgagctcggtacccatcagccaaaaagcatgcctgccacacaacatcaatttctggaaaacgctacacttaattatttctagtagaacagctctttggtttgccaaaaagaatcacctatagtggcatctaagcacaaaaaggagaaaaaaatcacaaagaaatgattgagaggcataataaaaattatcaaaaaattatgagttttacgatttcatctttttccaagttgaaatcatagggtggctttaacacagtgacaaggaatgtgcatgctgccattatggtgctctgcctaaaatggttggagccttgtcatgctacagagaaactgtcatacagcagggggtgccaaatttccatatttttttatatcattgagcaggtgcacagaagaccagaaagcactttctatcaggctggccttcctcttcctttccagtatgaagcaaaaactgccaatgaaactagcaattgttaaattcctttttcaaacagtatttgtgctatcagaacatagtgcattcaaaagtctagcctgagagaacaacccagttttattcattcctcctactacctctctcattcccactgtttgtgttctccctcccattttaattgtctatctagtccaaactaagcacacgatccagtccacattaaacaacatgttttcactttaagtcaaatacaagacacctttaatatcagcccttgttcataatcgtgcttctagtgacttaatgtacatgtcacactgtactgttgggttttgtgtctcatcatgaacaatgttgtgaaggtattaagtggagagtaagcagaattagattcctctaatgatgcacacccacactaagagcagaaataatattaaaaatagaaaaaaaagttttacatgagatttcaaatacccaggtatgagctgcagtttcttcaagttaaagcatcgaggttgtcagttacactattacaggaaacatatgcagagtttttattttagtatattagttttcacatatgtggaattactattaaactattctttcttttcaaatgcttaccattgtaaatgagtttgtgactttgtgtaggtgagtgcacatgactctggatgcctaagaggactgaagaagttggagttataggtagttttattctacttgactgttcagtgctaaaaatacaactgaggtcctttaaactgctgttcatgaacttcttaattgatatatctcatgagatctctaaactatttttattatgacacgtttcaccattttcactgtaacgatttttatgttttatattaatgtaactatatgacacttcccaaaatccccatattcacaattgaactgtttcaaagttttaccttgacttatgggaaatgaaaacccacattttataattttaaaatgaaatgtttattttatatttctgcaaatttcacaaggaaagattagtcactggtgtgtgagagcagaggagcataagagttcaggaatagaatccattatgattctggaggcaaggaagaactgatgccaaggtttcagtataagagcagtatccactggaaaggataaagtcactacatctgagcacagagcaggacatctacataatgagtggtcactaatgggccactgttacactgttatatgtataaggctcaagaatgagcactgaggctgtaaggtgtatgggtgaggacatcaggatgtaaacccagctcaggtagaggactcagaggacagcacagtcagcatgaactaataaacatcagataagataaggcacaagctcagctatatagggtaagggatctttgtaaatctgattgtgcatccagtctagttcaatgtgacttaggaagcccagtcatatgcaaatctagagaagactttagagtagaaatctgaggctcacctcacataccagcaagcgagtgaccagttagtcttaaggcaccacttcttagacatcatggcttgggtgtggaccttgccattcctgatggcagctgcccaaagtaagacatcagaaaaaagagttccaaggggaattgaagcagttccatgaatactcaccttcctgtgttcttttcacaggtgtccaggcacaggtgcagctggtggagtcaggagccgaagtgaaaaagcctggggcttcagtgaaggtgtcctgcaaggcctctggatacacattcactaattatattatccactgggtgaagcaggagcctggtcagggccttgaatggattggatattttaatccttacaatcatggtactaagtacaatgagaagttcaaaggcagggccacactaactgcagacaaatccatcagcacagcctacatggagctcagcagcctgcgctctgaggacactgcggtctactactgtgcaagatcaggaccctatgcctggtttgacacctggggccaagggaccacggtcaccgtctcctcaggtaagaatggccactctagggcctttgttttctgctgctgcctgtgggatttcatgagcattgcaaagttgtcctcgggacatgttccgaggggacctgggcggactggccaggaggggacgggcactggggtgccttgaggatctgggagcctctgtggattttccgatgcctttggaaaatgggactgaggttgggtgcgtctgagacagtaactcagcctgggggcttggtgaagatcgccgcacagcagcgagtccgtgaaatatcttatttagacttgtgaggtgcgctgtgtgtcaatttacatcttaaatcctttattggctggaaagagaattgttggagtgggtgaatccagccaggagggacgcggggggatcca

SEQ ID NO: 39 - DNA, 8096 bp, Artificial sequence
  Source 1..8096: /note= ”Description of artificial sequence Nucleotide sequence of expression vector LCVL2Sp20“
ctagagtcctagagaggtctggtggagcctgcaaaagtccagctttcaaaggaacacagaagtatgtgtatggaatattagaagatgttgcttttactcttaagttggttcctaggaaaaatagttaaatactgtgactttaaaatgtgagagggttttcaagtactcatttttttaaatgtccaaaatttttgtcaatcaatttgaggtcttgtttgtgtagaactgacattacttaaagtttaaccgaggaatgggagtgaggctctctcataccctattcagaactgacttttaacaataataaattaagtttaaaatatttttaaatgaattgagcaatgttgagttggagtcaagatggccgatcagaaccagaacacctgcagcagctggcaggaagcaggtcatgtggcaaggctatttggggaagggaaaataaaaccactaggtaaacttgtagctgtggtttgaagaagtggttttgaaacactctgtccagccccaccaaaccgaaagtccaggctgagcaaaacaccacctgggtaatttgcatttctaaaataagttgaggattcagccgaaactggagaggtcctcttttaacttattgagttcaaccttttaattttagcttgagtagttctagtttccccaaacttaagtttatcgacttctaaaatgtatttagaactcattttcaaaattaggttatgtaagaaattgaaggactttagtgtctttaatttctaatatatttagaaaacttcttaaaattactctattattcttccctctgattattggtctccattcaattcttttccaatacccgaagcatttacagtgactttgttcatgatcttttttagttgtttgttttgccttactattaagactttgacattctggtcaaaacggcttcacaaatctttttcaagaccactttctgagtattcattttaggagaaatactttttttttaaatgaatgcaattatctaggacctgcaggcatgctgttttctgtctgtccctaacatgccctgtgattatccgcaaacaacacacccaagggcagaactttgttacttaaacaccatcctgtttgcttctttcctcaggaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagagggagaagtgcccccacctgctcctcagttccagcctgaccccctcccatcctttggcctctgaccctttttccacaggggacctacccctattgcggtcctccagctcatctttcacctcacccccctcctcctccttggctttaattatgctaatgttggaggagaatgaataaataaagtgaatctttgcacctgtggtttctctctttcctcatttaataattattatctgttgttttaccaactactcaatttctcttataagggactaaatatgtagtcatcctaaggcgggatatcgagatctgaagctgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctagtcaaggcactatacatcaaatattccttattaacccctttacaaattaaaaagctaaaggtacacaatttttgagcatagttattaatagcagacactctatgcctgtgtggagtaagaaaaaacagtatgttatgattataactgttatgcctacttataaaggttacagaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagatccggaccaaagcggccatcgtgcctccccactcctgcagttcgggggcatggatgcgcggatagccgctgctggtttcctggatgccgacggatttgcactgccggtagaactccgcgaggtcgtccagcctcaggcagcagctgaaccaactcgcgaggggatcgagcatcccccatggtcttataaaaatgcatagctttaggaggggagcagagaacttgaaagcatcttcctgttagtctttcttctcgtagacttcaaacttatacttgatgcctttttcctcctggacctcagagaggacgcctgggtattctgggagaagtttatatttccccaaatcaatttctgggaaaaacgtgtcactttcaaattcctgcatgatccttgtcacaaagagtctgaggtggcctggttgattcatggcttcctggtaaacagaactgcctccgactatccaaaccatgtctactttacttgccaattccggttgttcaataagtcttaaggcatcatccaaacttttggcaagaaaatgagctcctcgtggtggttctttgagttctctactgagaactatattaattctgtcctttaaaggtcgattcttctcaggaatggagaaccaggttttcctacccataatcaccagattctgtttaccttccactgaagaggttgtggtcattctttggaagtacttgaactcgttcctgagcggaggccagggtcggtctccgttcttgccaatccccatattttgggacacggcgacgatgcagttcaatggtcgaaccatgatggcagcggggataaaatcctaccagccttcacgctaggattgccgtcaagtttgggggtaccgagctcgaattagctttttgcaaaagcctaggcctccaaaaaagcctcctcactacttctggaatagctcagagggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggccgcatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcagctgctcgaggaagagctcaaacccatgctactctctggcttgatggaagcaacgctttcatagctgagctgtcataaataataaagagatttttttattaatattgaaaagatgggttatttatgtaagactctgtcttcattttaaaaaccacaccttccagtagtattctgttactgttctggcaatcactgtgatcaagaagctacacggtgagttgtgcttctcagtcctaagggatacatctacaagaggctcccatactcgaagctcaggaaacattgtagaaaaggaggcaaaagactgacagagccagaggaccaagaaatttgttgtgaggttgtgtctcctactaacaatataagcaatatctataaattgttgatatcatggctactaaaatgtgagttgaacgaggaggacacaaatgaacatgacaatcagaatgaggcctctcacctgcaaaaaacactatagagaagcagataaagctgtcagcagaagaggcgcacctccttatagaagaagcctaccaggtttgatatatcagccttgaaaacctacatagtatttacattatatcgagtctatgagacatatttagtaatgcatatgtatgtgtgtgtgtgcatgtatgtgtgtaaatacatatgttcatagaaaaatgtgtaaaaagagatcatgaatttaagagagaactgggacaatttttttcagggagttgtaatcaggaaagttaagggaaaaatgttgtaattaaaattcaggctcagaaacaaacaaaggaaaagaaaaaaaaacaacaacaacaacaaaaaaacaaaacaaaggagaagctgtatggccacaatagcatctacagctaactgtgaaaggataatggaacaagttatgtactgcctagagcagtatgatgcctaaatcatctcgacatggaggaaaatagaacaaagacactctacatagactatgatagaaatcaaaataaggtgtaagacatagaacattagttttgtttgttgttcaaagagactcacattcccacaaaaaaatctgtgggattttacaggtctgcaataagctgctgacctgatgatttctgcagctgtgcctaccctttgctgatttgcatgtacccaaagcatagcttactgacatgaggatttcttcatagtcaggtcacaccctttgctggagtcagaatcacactgatcacacacagtcatgagtgtgctcactcaggtcctggcgttgctgctgctgtggcttacaggtaatgaagacagcactagaattttattgagcttcctgtacactgtgctgcttgtctctgtgaaaattctcttgtgaattaatcatgtggggatctgttttcaatttttcaattgtaggtacgcgttgtgacattctgctgacccagtctccagccaccctgtctctgagtccaggagaaagagccactttctcctgcagggccagtcagaacattggcacaagcatacagtggtatcaacaaaaaacaaatggtgctccaaggcttctcataaggtcttcttctgagtctatctctgggatcccttccaggtttagtggcagtggatcagggacagattttactcttaccatcagcagtctggagcctgaagattttgcagtgtattactgtcaacaaagtaatacctggccattcacgttcggccaggggaccaagcttgaaatcaaacgtaagtagaatccaaagtctctttcttccgttgtctatgtctgtggcttctatgtctaaaaatgatgtataaaatcttactctgaaaccagattctggcactctccaaggcaaagatacagagtaactccgtaagcaaagctgggaataggctagacatgttctctggagaatgaatgccagtgtaataattaacacaagtgatagtttcagaaatgctctagtt

SEQ ID NO: 40 - DNA, 11563 bp, Artificial sequence
  Source 1..11563: /note= ”Description of artificial sequence Nucleotide sequence of expression vector HCVHESp20“
ctagagaggtctggtggagcctgcaaaagtccagctttcaaaggaacacagaagtatgtgtatggaatattagaagatgttgcttttactcttaagttggttcctaggaaaaatagttaaatactgtgactttaaaatgtgagagggttttcaagtactcatttttttaaatgtccaaaatttttgtcaatcaatttgaggtcttgtttgtgtagaactgacattacttaaagtttaaccgaggaatgggagtgaggctctctcataccctattcagaactgacttttaacaataataaattaagtttaaaatatttttaaatgaattgagcaatgttgagttggagtcaagatggccgatcagaaccagaacacctgcagcagctggcaggaagcaggtcatgtggcaaggctatttggggaagggaaaataaaaccactaggtaaacttgtagctgtggtttgaagaagtggttttgaaacactctgtccagccccaccaaaccgaaagtccaggctgagcaaaacaccacctgggtaatttgcatttctaaaataagttgaggattcagccgaaactggagaggtcctcttttaacttattgagttcaaccttttaattttagcttgagtagttctagtttccccaaacttaagtttatcgacttctaaaatgtatttaagctttctggggcaggccaggcctgaccttggctttggggcagggagggggctaaggtgaggcaggtggcgccagccaggtgcacacccaatgcccatgagcccagacactggacgctgaacctcgcggacagttaagaacccaggggcctctgcgccctgggcccagctctgtcccacaccgcggtcacatggcaccacctctcttgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttggtgagaggccagcacagggagggagggtgtctgctggaagccaggctcagcgctcctgcctggacgcatcccggctatgcagtcccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatgagtgcgacggccggcaagcccccgctccccgggctctcgcggtcgcacgaggatgcttggcacgtaccccgtctacatacttcccaggcacccagcatggaaataaagcacccaccactgccctgggcccctgcgagactgtgatggttctttccacgggtcaggccgagtctgaggcctgagtggcatgagggaggcagagcgggtcccactgtccccacactggcccaggctgtgcaggtgtgcctgggccgcctagggtggggctcagccaggggctgccctcggcagggtgggggatttgccagcgtggccctccctccagcagcagctgccctgggctgggccacgagaagccctaggagcccctggggacagacacacagcccctgcctctgtaggagactgtcctgtcctgtgagcgccctgtcctccgaccccgatgcccactcgggggcatgcctagtccatgcgcgtagggacaggccctccctcacccatctacccccacggcactaacccctggcagccctgcccagcctcgcacccgcatggggacacaaccgactccggggacatgcactctcgggccctgtggagggactggtgcagatgcccacacacacactcagcccagacccgttcaacaaaccccgcactgaggttggtcgagcgggagtgcggccagagcctgcctcggccgtcagggaggactcccgggctcactcgaaggaggtgccaccatttcagctttggtagcttttcttcttcttttaaattttctaaagctcattaattgtctttgatgtttcttttgtgatgacaataaaatatcctttttaagtcttgtacttcgtgatgggagccgccttcctgtgtccacgcgcctcctgcccccggtgggaagcacggtcaggaggaggctggtccagctgcacctcgggggctccctgcactcgccccccgcctcctgcagccacacgcattgcccgagcgaccctccctggcccctgtcactacatggacccctggggcttctcctcttttctacatggatgcagtttctcctcctgctgggcacggtgctgcctgccctggtcactctgcgggggacagggcctccagggaaagctgggtcgaggctgggagctggctcaggctggccaggcagagccacagggagggccttccagaaccaaccatggtccgaagcgagaggtgggtgtcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctagtcaaggcactatacatcaaatattccttattaacccctttacaaattaaaaagctaaaggtacacaatttttgagcatagttattaatagcagacactctatgcctgtgtggagtaagaaaaaacagtatgttatgattataactgttatgcctacttataaaggttacagaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagatccggaccaaagcggccatcgtgcctccccactcctgcagttcgggggcatggatgcgcggatagccgctgctggtttcctggatgccgacggatttgcactgccggtagaactccgcgaggtcgtccagcctcaggcagcagctgaaccaactcgcgaggggatcgagcccggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcgagctcggtacccatcagccaaaaagcatgcctgccacacaacatcaatttctggaaaacgctacacttaattatttctagtagaacagctctttggtttgccaaaaagaatcacctatagtggcatctaagcacaaaaaggagaaaaaaatcacaaagaaatgattgagaggcataataaaaattatcaaaaaattatgagttttacgatttcatctttttccaagttgaaatcatagggtggctttaacacagtgacaaggaatgtgcatgctgccattatggtgctctgcctaaaatggttggagccttgtcatgctacagagaaactgtcatacagcagggggtgccaaatttccatatttttttatatcattgagcaggtgcacagaagaccagaaagcactttctatcaggctggccttcctcttcctttccagtatgaagcaaaaactgccaatgaaactagcaattgttaaattcctttttcaaacagtatttgtgctatcagaacatagtgcattcaaaagtctagcctgagagaacaacccagttttattcattcctcctactacctctctcattcccactgtttgtgttctccctcccattttaattgtctatctagtccaaactaagcacacgatccagtccacattaaacaacatgttttcactttaagtcaaatacaagacacctttaatatcagcccttgttcataatcgtgcttctagtgacttaatgtacatgtcacactgtactgttgggttttgtgtctcatcatgaacaatgttgtgaaggtattaagtggagagtaagcagaattagattcctctaatgatgcacacccacactaagagcagaaataatattaaaaatagaaaaaaaagttttacatgagatttcaaatacccaggtatgagctgcagtttcttcaagttaaagcatcgaggttgtcagttacactattacaggaaacatatgcagagtttttattttagtatattagttttcacatatgtggaattactattaaactattctttcttttcaaatgcttaccattgtaaatgagtttgtgactttgtgtaggtgagtgcacatgactctggatgcctaagaggactgaagaagttggagttataggtagttttattctacttgactgttcagtgctaaaaatacaactgaggtcctttaaactgctgttcatgaacttcttaattgatatatctcatgagatctctaaactatttttattatgacacgtttcaccattttcactgtaacgatttttatgttttatattaatgtaactatatgacacttcccaaaatccccatattcacaattgaactgtttcaaagttttaccttgacttatgggaaatgaaaacccacattttataattttaaaatgaaatgtttattttatatttctgcaaatttcacaaggaaagattagtcactggtgtgtgagagcagaggagcataagagttcaggaatagaatccattatgattctggaggcaaggaagaactgatgccaaggtttcagtataagagcagtatccactggaaaggataaagtcactacatctgagcacagagcaggacatctacataatgagtggtcactaatgggccactgttacactgttatatgtataaggctcaagaatgagcactgaggctgtaaggtgtatgggtgaggacatcaggatgtaaacccagctcaggtagaggactcagaggacagcacagtcagcatgaactaataaacatcagataagataaggcacaagctcagctatatagggtaagggatctttgtaaatctgattgtgcatccagtctagttcaatgtgacttaggaagcccagtcatatgcaaatctagagaagactttagagtagaaatctgaggctcacctcacataccagcaagcgagtgaccagttagtcttaaggcaccacttcttagacatcatggcttgggtgtggaccttgccattcctgatggcagctgcccaaagtaagacatcagaaaaaagagttccaaggggaattgaagcagttccatgaatactcaccttcctgtgttcttttcacaggtgtccaggcagaggtgcagctggtggagtcaggagccgaagtgaaaaagcctggggcttcagtgaaggtgtcctgcaaggcctctggatacacattcactaattatattatccactgggtgaagcaggagcctggtcagggccttgaatggattggatattttaatccttacaatcatggtactaagtacaatgagaagttcaaaggcagggccacactaactgcaaacaaatccatcagcacagcctacatggagctcagcagcctgcgctctgaggacactgcggtctactactgtgcaagatcaggaccctatgcctggtttgacacctggggccaagggaccacggtcaccgtctcctcaggtaagaatggccactctagggcctttgttttctgctgctgcctgtgggatttcatgagcattgcaaagttgtcctcgggacatgttccgaggggacctgggcggactggccaggaggggacgggcactggggtgccttgaggatctgggagcctctgtggattttccgatgcctttggaaaatgggactgaggttgggtgcgtctgagacagtaactcagcctgggggcttggtgaagatcgccgcacagcagcgagtccgtgaaatatcttatttagacttgtgaggtgcgctgtgtgtcaatttacatcttaaatcctttattggctggaaagagaattgttggagtgggtgaatccagccaggagggacgcggggggatcca

SEQ ID NO: 41 - DNA, 11563 bp, Artificial sequence
  Source 1..11563: /note= ”Description of artificial sequence Nucleotide sequence of expression vector HCVHQSp20“
ctagagaggtctggtggagcctgcaaaagtccagctttcaaaggaacacagaagtatgtgtatggaatattagaagatgttgcttttactcttaagttggttcctaggaaaaatagttaaatactgtgactttaaaatgtgagagggttttcaagtactcatttttttaaatgtccaaaatttttgtcaatcaatttgaggtcttgtttgtgtagaactgacattacttaaagtttaaccgaggaatgggagtgaggctctctcataccctattcagaactgacttttaacaataataaattaagtttaaaatatttttaaatgaattgagcaatgttgagttggagtcaagatggccgatcagaaccagaacacctgcagcagctggcaggaagcaggtcatgtggcaaggctatttggggaagggaaaataaaaccactaggtaaacttgtagctgtggtttgaagaagtggttttgaaacactctgtccagccccaccaaaccgaaagtccaggctgagcaaaacaccacctgggtaatttgcatttctaaaataagttgaggattcagccgaaactggagaggtcctcttttaacttattgagttcaaccttttaattttagcttgagtagttctagtttccccaaacttaagtttatcgacttctaaaatgtatttaagctttctggggcaggccaggcctgaccttggctttggggcagggagggggctaaggtgaggcaggtggcgccagccaggtgcacacccaatgcccatgagcccagacactggacgctgaacctcgcggacagttaagaacccaggggcctctgcgccctgggcccagctctgtcccacaccgcggtcacatggcaccacctctcttgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttggtgagaggccagcacagggagggagggtgtctgctggaagccaggctcagcgctcctgcctggacgcatcccggctatgcagtcccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatgagtgcgacggccggcaagcccccgctccccgggctctcgcggtcgcacgaggatgcttggcacgtaccccgtctacatacttcccaggcacccagcatggaaataaagcacccaccactgccctgggcccctgcgagactgtgatggttctttccacgggtcaggccgagtctgaggcctgagtggcatgagggaggcagagcgggtcccactgtccccacactggcccaggctgtgcaggtgtgcctgggccgcctagggtggggctcagccaggggctgccctcggcagggtgggggatttgccagcgtggccctccctccagcagcagctgccctgggctgggccacgagaagccctaggagcccctggggacagacacacagcccctgcctctgtaggagactgtcctgtcctgtgagcgccctgtcctccgaccccgatgcccactcgggggcatgcctagtccatgcgcgtagggacaggccctccctcacccatctacccccacggcactaacccctggcagccctgcccagcctcgcacccgcatggggacacaaccgactccggggacatgcactctcgggccctgtggagggactggtgcagatgcccacacacacactcagcccagacccgttcaacaaaccccgcactgaggttggtcgagcgggagtgcggccagagcctgcctcggccgtcagggaggactcccgggctcactcgaaggaggtgccaccatttcagctttggtagcttttcttcttcttttaaattttctaaagctcattaattgtctttgatgtttcttttgtgatgacaataaaatatcctttttaagtcttgtacttcgtgatgggagccgccttcctgtgtccacgcgcctcctgcccccggtgggaagcacggtcaggaggaggctggtccagctgcacctcgggggctccctgcactcgccccccgcctcctgcagccacacgcattgcccgagcgaccctccctggcccctgtcactacatggacccctggggcttctcctcttttctacatggatgcagtttctcctcctgctgggcacggtgctgcctgccctggtcactctgcgggggacagggcctccagggaaagctgggtcgaggctgggagctggctcaggctggccaggcagagccacagggagggccttccagaaccaaccatggtccgaagcgagaggtgggtgtcagatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatctctagtcaaggcactatacatcaaatattccttattaacccctttacaaattaaaaagctaaaggtacacaatttttgagcatagttattaatagcagacactctatgcctgtgtggagtaagaaaaaacagtatgttatgattataactgttatgcctacttataaaggttacagaatatttttccataattttcttgtatagcagtgcagctttttcctttgtggtgtaaatagcaaagcaagcaagagttctattactaaacacagcatgactcaaaaaacttagcaattctgaaggaaagtccttggggtcttctacctttctcttcttttttggaggagtagaatgttgagagtcagcagtagcctcatcatcactagatggcatttcttctgagcaaaacaggttttcctcattaaaggcattccaccactgctcccattcatcagttccataggttggaatctaaaatacacaaacaattagaatcagtagtttaacacattatacacttaaaaattttatatttaccttagagctttaaatctctgtaggtagtttgtccaattatgtcacaccacagaagtaaggttccttcacaaagatccggaccaaagcggccatcgtgcctccccactcctgcagttcgggggcatggatgcgcggatagccgctgctggtttcctggatgccgacggatttgcactgccggtagaactccgcgaggtcgtccagcctcaggcagcagctgaaccaactcgcgaggggatcgagcccggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaagaattcgagctcggtacccatcagccaaaaagcatgcctgccacacaacatcaatttctggaaaacgctacacttaattatttctagtagaacagctctttggtttgccaaaaagaatcacctatagtggcatctaagcacaaaaaggagaaaaaaatcacaaagaaatgattgagaggcataataaaaattatcaaaaaattatgagttttacgatttcatctttttccaagttgaaatcatagggtggctttaacacagtgacaaggaatgtgcatgctgccattatggtgctctgcctaaaatggttggagccttgtcatgctacagagaaactgtcatacagcagggggtgccaaatttccatatttttttatatcattgagcaggtgcacagaagaccagaaagcactttctatcaggctggccttcctcttcctttccagtatgaagcaaaaactgccaatgaaactagcaattgttaaattcctttttcaaacagtatttgtgctatcagaacatagtgcattcaaaagtctagcctgagagaacaacccagttttattcattcctcctactacctctctcattcccactgtttgtgttctccctcccattttaattgtctatctagtccaaactaagcacacgatccagtccacattaaacaacatgttttcactttaagtcaaatacaagacacctttaatatcagcccttgttcataatcgtgcttctagtgacttaatgtacatgtcacactgtactgttgggttttgtgtctcatcatgaacaatgttgtgaaggtattaagtggagagtaagcagaattagattcctctaatgatgcacacccacactaagagcagaaataatattaaaaatagaaaaaaaagttttacatgagatttcaaatacccaggtatgagctgcagtttcttcaagttaaagcatcgaggttgtcagttacactattacaggaaacatatgcagagtttttattttagtatattagttttcacatatgtggaattactattaaactattctttcttttcaaatgcttaccattgtaaatgagtttgtgactttgtgtaggtgagtgcacatgactctggatgcctaagaggactgaagaagttggagttataggtagttttattctacttgactgttcagtgctaaaaatacaactgaggtcctttaaactgctgttcatgaacttcttaattgatatatctcatgagatctctaaactatttttattatgacacgtttcaccattttcactgtaacgatttttatgttttatattaatgtaactatatgacacttcccaaaatccccatattcacaattgaactgtttcaaagttttaccttgacttatgggaaatgaaaacccacattttataattttaaaatgaaatgtttattttatatttctgcaaatttcacaaggaaagattagtcactggtgtgtgagagcagaggagcataagagttcaggaatagaatccattatgattctggaggcaaggaagaactgatgccaaggtttcagtataagagcagtatccactggaaaggataaagtcactacatctgagcacagagcaggacatctacataatgagtggtcactaatgggccactgttacactgttatatgtataaggctcaagaatgagcactgaggctgtaaggtgtatgggtgaggacatcaggatgtaaacccagctcaggtagaggactcagaggacagcacagtcagcatgaactaataaacatcagataagataaggcacaagctcagctatatagggtaagggatctttgtaaatctgattgtgcatccagtctagttcaatgtgacttaggaagcccagtcatatgcaaatctagagaagactttagagtagaaatctgaggctcacctcacataccagcaagcgagtgaccagttagtcttaaggcaccacttcttagacatcatggcttgggtgtggaccttgccattcctgatggcagctgcccaaagtaagacatcagaaaaaagagttccaaggggaattgaagcagttccatgaatactcaccttcctgtgttcttttcacaggtgtccaggcacaggtgcagctggtggagtcaggagccgaagtgaaaaagcctggggcttcagtgaaggtgtcctgcaaggcctctggatacacattcactaattatattatccactgggtgaagcaggagcctggtcagggccttgaatggattggatattttaatccttacaatcatggtactaagtacaatgagaagttcaaaggcagggccacactaactgcaaacaaatccatcagcacagcctacatggagctcagcagcctgcgctctgaggacactgcggtctactactgtgcaagatcaggaccctatgcctggtttgacacctggggccaagggaccacggtcaccgtctcctcaggtaagaatggccactctagggcctttgttttctgctgctgcctgtgggatttcatgagcattgcaaagttgtcctcgggacatgttccgaggggacctgggcggactggccaggaggggacgggcactggggtgccttgaggatctgggagcctctgtggattttccgatgcctttggaaaatgggactgaggttgggtgcgtctgagacagtaactcagcctgggggcttggtgaagatcgccgcacagcagcgagtccgtgaaatatcttatttagacttgtgaggtgcgctgtgtgtcaatttacatcttaaatcctttattggctggaaagagaattgttggagtgggtgaatccagccaggagggacgcggggggatcca